(19) United States
(12) Patent Application Publication
(10) Pub. No.: US 2024/0408087 A1
(43) Pub. Date: Dec. 12, 2024

(54) TRPC6 INHIBITORY COMPOUNDS FOR TREATING SEPSIS

(71) Applicant: Boehringer Ingelheim International GmbH, Ingelheim am Rhein (DE)

(72) Inventors: Thierry BOUYSSOU, Warthausen (DE); Paul NICKLIN, Biberach an der Riss (DE)

(21) Appl. No.: 18/700,748

(22) PCT Filed: Oct. 14, 2022
(86) PCT No.: PCT/EP2022/078609

§ 371 Date: Apr. 12, 2024

(30) Foreign Application Priority Data
Oct. 15, 2021 (EP) 21202812.0

(51) Int. Cl. A61K 31/501; A61P 29/00

(52) U.S. Cl. CPC A61K 31/501; A61P 29/00

(57) ABSTRACT

The present invention relates to compounds of formula (I)

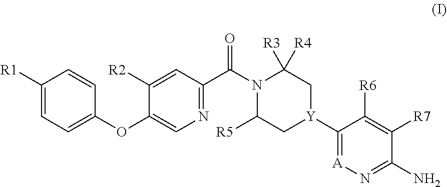

for use in methods for of a patient with a disorder associated with bacterial or fungal severe sepsis, and bacterial or fungal septic shock and conditions arising therefrom, wherein the groups Y, A and R1 to R7 are as defined herein, pharmaceutical compositions which contain compounds of this kind and their use as medicaments for the treatment of bacterial or fungal severe sepsis and bacterial or fungal septic shock and conditions arising therefrom.

### FIELD OF THE INVENTION

The present invention relates to compounds for use in a method for treatment of a patient with a systemic response to bacteria, fungi or circulating bacterial or fungal products and conditions arising therefrom.

### BACKGROUND OF THE INVENTION

In bacterial or fungal severe sepsis or bacterial or fungal septic shock increase in vascular permeability increases in several organs including but not limited to the lung, kidney, liver and heart. Interstitial fluid accumulation in these organs impairs their proper functioning (e.g. causing hypovolemia, hypotension, arrhythmia, glomerular filtration disruption, or impairment of the metabolism) and leads to organ failure followed by death. Regular antibiotics are not used for fungal infections because they are not effective.

Sepsis, severe sepsis, and septic shock are disorders arising from the systemic inflammatory response to an infection (see Mitchell M. Levy et al., Crit Care Med. 2003 April; 31(4):1250-6.). Sepsis is a disorder having both an infection (e.g., bacterial, fungal, abdominal trauma, gut perforation) and a systemic inflammatory response. This leads to increase in vascular permeability of several organs such as kidney liver, heart and lung. Severe sepsis (sepsis with organ dysfunction) refers to sepsis with acute organ dysfunction caused by sepsis. Septic shock refers to persistent hypotension unexplained by other causes.

There is a need for compounds which can be used in a method for treating bacterial or fungal severe sepsis and bacterial or fungal septic shock.

### SUMMARY OF THE INVENTION

The present invention provides compounds for use in a method for treatment of a patient with bacterial or fungal severe sepsis and bacterial or fungal septic shock and conditions arising therefrom, in particular conditions associated with bacterial or fungal parasites infections.

In one embodiment the invention relates to a pharmaceutical composition for use in a method for treatment of a patient with a systemic inflammatory response to a bacterial or fungal infection such as severe sepsis and bacterial or fungal septic shock arising therefrom, comprising and administering to the patient in need thereof a pharmaceutically effective amount of a compound of formula (I),

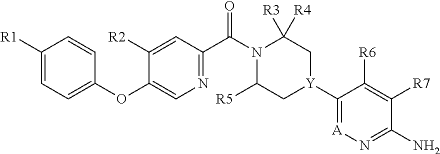

wherein
Y is CH or N;
A is CH or N;
R1 is selected from the group consisting of methyl, ethyl and propyl in which the hydrogen atoms may be partially or fully replaced by fluorine,
or R1 is selected from the group consisting of halogen, C₃₋₆-cycloalkyl, OC₃₋₆-cycloalkyl, and OC₁₋₆-alkyl wherein the alkyl groups may optionally be substituted with 1 to 3 halogen and C₃₋₆-cycloalkyl optionally substituted with 1 to 3 groups independently selected from the group consisting of halogen and C₁₋₆-alkyl optionally substituted with 1 to 3 halogen,
R2 is selected from the group consisting of H, C₁₋₆-alkyl, OCF₃, C₃₋₆-cycloalkyl, OC₁₋₆-alkyl, and OC₃₋₆-cycloalkyl,
R3 is selected from the group consisting of H, C₁₋₆-alkyl, C₃₋₆-cycloalkyl, and OC₃₋₆-cycloalkyl; wherein each of the C₁₋₆-alkyl, C₃₋₆-cycloalkyl, OC₃₋₆-cycloalkyl of the R3 group may be optionally substituted with one to three groups each independently selected from the group consisting of halogen, OH, OC₁₋₆-alkyl, SC₁₋₆-alkyl, and N(C₁₋₆-alky)₂; and
wherein one to three carbon atoms of the C₁₋₆-alkyl of the R3 group may optionally be replaced with one or two moieties selected from the group consisting of NH, N(C₁₋₆-alkyl), O, and S;
R4 and R5 are each independently selected from the group consisting of H and C₁₋₆-alkyl;
R3 and R4 can together with the atom to which they are attached join to form a 3 to 9-membered carbocyclyl ring which optionally may contain one to three heteroatoms selected from the group consisting of N, O, and S or
R3 and R5 can together with the atoms to which they are attached join to form a 3 to 9-membered bicyclic ring which optionally may contain one to three heteroatoms selected from the group consisting of N, O, and S;
R6 is selected from the group consisting of H, C₁₋₆-alkyl, CN, CF₃, OCF₃, C₃₋₆-cycloalkyl, OC₁₋₆-alkyl, and OC₃₋₆-cycloalkyl,
R7 is selected from the group consisting of H and OC₁₋₆-alkyl,
or a pharmaceutically acceptable salt thereof together with one or more pharmaceutically acceptable carrier.

In a particular embodiment, the above invention relates to a pharmaceutical composition, wherein the patient's bacterial or fungal severe sepsis and septic shock is associated with ARDS, related to infection.

In another embodiment the invention relates to a pharmaceutical composition comprising a compound of formula (I) for use in a method for treatment of a patient with a systemic inflammatory response to a bacterial or fungal infection such as severe sepsis and bacterial or fungal septic shock and/or conditions arising therefrom, wherein
R1 of formula (I) is selected from the group consisting of CF₃, halogen, OC₃₋₆-cycloalkyl, and OC₁₋₆-alkyl optionally substituted with one to three halogen and C₃₋₆-cycloalkyl optionally substituted with 1 to 3 halogen groups,
R2 of formula (I) is OC₁₋₆-alkyl,
R3 of formula (I) is selected from the group consisting of H and C₁₋₆-alkyl optionally substituted with OH or OC₁₋₆-alkyl,
R4 of formula (I) is H,
R5 of formula (I) is H,
R3 and R4 of formula (I) can together with the atom to which they are attached join to form a 3 to 9-membered carbocyclyl ring which optionally may contain one to three heteroatoms selected from the group consisting of N and O; or
R3 and R5 of formula (I) can together with the atoms to which they are attached join to form a 3 to 9-membered bicyclic which optionally may contain one to three heteroatoms selected from the group consisting of N and O;
R6 of formula (I) is selected from the group consisting of H, C₁₋₆-alkyl, OC₁₋₆-alkyl, and OC₃₋₆-cycloalkyl,
R7 of formula (I) is selected from the group consisting of H and OC₁₋₆-alkyl such as methoxy,
or a pharmaceutically acceptable salt thereof.

In a further embodiment the invention relates to a pharmaceutical composition comprising a compound of formula (I) for use in a method for treatment of a patient with bacterial or fungal severe sepsis and bacterial or fungal septic shock and/or conditions arising therefrom, wherein
A is CH and Y is N, or
A is CH and Y is CH, or
A is N and Y is CH,
or a pharmaceutically acceptable salt thereof.

In yet a further embodiment the invention relates to a pharmaceutical composition comprising a compound of formula (I) for use in a method for treatment of a patient with bacterial or fungal severe sepsis and bacterial or fungal septic shock and/or conditions arising therefrom, wherein the compound of formula (I) has the structure

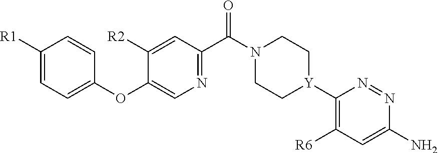

wherein
Y is CH or N,
R1 is selected from the group consisting of CF₃, halogen, OC₃₋₆-cycloalkyl, and OC₁₋₆-alkyl optionally substituted with one to three halogen and unsubstituted cyclohexyl or cyclohexyl substituted with a group selected from the group consisting of fluorine (F), unsubstituted —CH₃, —CH₃ substituted with 1-3 fluoro atoms, unsubstituted —CH₂CH₃, —CH₂CH₃ substituted with 1-5 fluoro atoms, unsubstituted propyl and propyl substituted with 1-7 fluoro atoms;
R2 is selected from the group consisting of H, —CH₃, —CH₂CH₃, -cyclopropyl, -cyclobutyl, -cyclopentyl, -cyclohexyl, and OC₁₋₆-alkyl such as —O—CH₃, —O—CH₂CH₃, O—CF₃, hydroxymethyl, hydroxyethyl and hydroxypropyl; and
R6 is selected from the group consisting of H (hydrogen), unsubstituted methyl, unsubstituted ethyl, unsubstituted propyl or methyl substituted with 1-3 fluoro atoms, ethyl substituted with 1-5 fluoro atoms and propyl substituted with 1-7 fluoro atoms;
R7 is selected from the group consisting of H and OC₁₋₆-alkyl,
or a pharmaceutically acceptable salt thereof.

In another embodiment the invention relates to a pharmaceutical composition comprising a compound of formula (I) having the structure

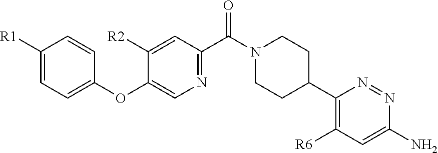

for use in a method for treatment of a patient with bacterial or fungal severe sepsis and bacterial or fungal septic shock and/or conditions arising therefrom, wherein
R1 is unsubstituted methyl, ethyl or propyl or methyl, ethyl or propyl substituted with 1-7 fluorine atoms, or fluorine,
R2 is OC₁₋₆-alkyl such as methoxy, ethoxy or propoxy,
R6 is selected from the group consisting of H, unsubstituted methyl, unsubstituted ethyl and unsubstituted propyl, methyl substituted with 1-3 fluoro atoms, ethyl substituted with 1-5 fluoro atoms, and propyl substituted with 1-7 fluoro atoms; methoxy, ethoxy, propoxy, and cyclylpropyloxy,
or a pharmaceutically acceptable salt thereof.

In another embodiment the invention relates to a pharmaceutical composition comprising a compound of formula (I) having the structure

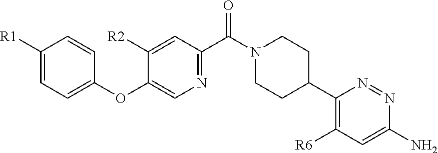

for use in a treatment of a patient with bacterial or fungal severe sepsis and bacterial or fungal septic shock and/or conditions arising therefrom, wherein
R1 is unsubstituted methyl, ethyl or propyl or methyl, ethyl or propyl substituted with 1-7 fluorine atoms, or fluorine,
R2 is selected from OC₁₋₆-alkyl such as methoxy, ethoxy and propoxy,
R6 is selected from the group consisting of H, unsubstituted methyl, unsubstituted ethyl and unsubstituted propyl, methyl substituted with 1-3 fluoro atoms, ethyl substituted with 1-5 fluoro atoms, and propyl substituted with 1-7 fluoro atoms; methoxy, ethoxy, propoxy and cyclylpropyloxy,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
Y is CH and A is N,
R1 represents Cl, F, methoxy, isopropoxy, trifluoromethyl, difluoromethoxy, cyclopropyloxy,
R2 is methoxy or ethoxy,
R3, R4 and R5 are each H,
R6 is H, methyl, methoxy or ethoxy,
R7 is H,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
Y is CH and A is CH,
R1, Cl, F, methoxy, trifluoromethyl,
R2 is methoxy or ethoxy,
R3, R4 and R5 are each H,
R6 is H, methyl, methoxy, or ethoxy,
R7 is H,
or a pharmaceutically acceptable salt thereof.

In another embodiment, the invention relates to a pharmaceutical composition comprising a compound of formula (I) for use in a method for treatment of a patient with bacterial or fungal severe sepsis and septic shock and/or conditions arising therefrom, wherein
Y is N and A is CH,
R1 represents H or fluoro,
R2 is methoxy,
R3 is selected from the group consisting of H, 2-hydroxymethyl, and hydroxyethyl,
R4 is H,
R5 is H,
R3 and R4 may join to form a spirocyclic ring, or
R3 and R5 may together with the atoms to which they are attached join to form a bicyclic ring,
R6 is selected from the group consisting of H and methoxy,
R7 is H,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
R1 is C₁₋₆-alkyl optionally substituted with 1 to 3 groups independently selected from the group consisting of halogen and C₃₋₆-cycloalkyl,
R2 is OC₁₋₆-alkyl,
R3, R4 and R5 are each H,
R6 is selected from the group consisting of H, C₁₋₆-alkyl, and OC₁₋₆-alkyl,
R7 is H,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
R1 represents a group selected from the group consisting of ethyl, propyl, isopropyl, isobutyl, cyclopropylmethyl, cyclobutylmethyl, 2,2-dimethylpropyl, 1-methylcyclopropylmethyl, 1-fluoromethylcyclopropylmethyl, 1-cyclopropylethyl, 2-cyclopropylethyl, cyclopentyl, cyclohexyl, 2,2-difluorocyclobutylmethyl, 3,3-difluorocyclobutylmethyl, 3-(trifluoromethyl)cyclobutylmethyl, and 3,3,3-trifluoro-2-methyl-propyl;
R2 is methoxy,
R3, R4 and R5 are each H,
R6 is selected from the group consisting of H, methyl, and methoxy,
R7 is H;
or a pharmaceutically acceptable salt thereof.

In another embodiment the invention relates to a pharmaceutical composition comprising compound of formula (I) for use in a method for treatment of a patient with bacterial or fungal severe sepsis and septic shock and/or conditions arising therefrom, wherein
Y is CH and A is N;
R1 represents a group selected from the group consisting of propyl, isopropyl, isobutyl, cyclopropylmethyl, cyclobutylmethyl, 2,2-dimethylpropyl, 1-cyclopropylethyl, 2-cyclopropylethyl, and cyclohexyl,
R2 is methoxy,
R3, R4 and R5 are each H,
R6 is selected from the group consisting of H, methyl, and methoxy,
R7 is H,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
Y is CH and A is CH,
R1 represents a group selected from the group consisting of ethyl, propyl, isopropyl, isobutyl, cyclopropylmethyl, cyclobutylmethyl, 2,2-dimethylpropyl, 1-methylcyclopropylmethyl, 1-fluoromethylcyclopropylmethyl, 1-cyclopropylethyl, 2-cyclopropylethyl, cyclopentyl, cyclohexyl, 2,2-difluorocyclobutylmethyl, 3,3-difluorocyclobutylmethyl, 3-(trifluoromethyl)cyclobutylmethyl, and 3,3,3-trifluoro-2-methyl-propyl,
R2 is methoxy,
R3, R4 and R5 are each H,
R6 is selected from the group consisting of H, methyl, and methoxy,
R7 is H,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
R3 and R4 together with the atom to which they are attached join to form a 3-membered carbocyclyl ring,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
R3 and R5 together with the atoms to which they are attached join to form a 3 to 9-membered bicyclic ring which optionally may contain one to two heteroatoms independently selected from the group consisting of N and O, and
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
Y is CH,
A is N,
R2 is OCH₃; and
R3, R4, R5 and R7 are each H,
or a pharmaceutically acceptable salt thereof.

In another embodiment the invention relates to a compound of formula (I) for use in a method for treatment of a patient with bacterial or fungal severe sepsis and septic shock and/or conditions arising therefrom, wherein
R1 is phenyl optionally substituted with 1 to 3 groups independently selected from the group consisting of methyl, ethyl or propyl or methyl, ethyl or propyl substituted with 1-7 fluorine atoms, CF₃, halogen, C₃₋₆-cycloalkyl, OC₃₋₆-cycloalkyl, and OC₁₋₆-alkyl optionally substituted with one to three halogen; and
R6 is H; or OCH₃,
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
R1 is selected from the group consisting of unsubstituted phenyl or phenyl substituted with 1 to 3 groups independently selected from the groups consisting of unsubstituted methyl, unsubstituted ethyl, unsubstituted propyl, methyl substituted with 1-3 fluorine atoms, ethyl substituted with 1-5 fluorine atoms and propyl substituted with 1-7 fluorine atoms, halogen, OC₃₋₆-cycloalkyl, and OC₁₋₆-alkyl optionally substituted with one to three halogen;
R2 is OCH₃ or OCH₂CH₃,
R3, R4, R5·R6, and R7 are each H; and
or a pharmaceutically acceptable salt thereof.

In another embodiment the invention relates to a pharmaceutical composition comprising a compound of formula (I) for use in a method for treatment of a patient with bacterial or fungal severe sepsis and septic shock and/or conditions arising therefrom, wherein
R1 is selected from the group consisting of unsubstituted phenyl or phenyl substituted with 1 to 3 groups independently selected from CF₃, halogen, OC₃₋₆-cycloalkyl, unsubstituted OC₁₋₆-alkyl and OC₁₋₆-alkyl substituted in the alkyl moiety with one to three halogen;
R2 is OCH₃ of OCH₂CH₃,
R3, R4, R5 and R7 are each H,
R6 is CH₃ or OCH₃,
Y is CH; and
A is N,
or a pharmaceutically acceptable salt thereof.

In another embodiment the invention relates to a pharmaceutical composition comprising a compound of formula (I) for use in a method treatment of a patient with bacterial or fungal severe sepsis and septic shock and/or conditions arising therefrom or a pharmaceutically acceptable salt thereof.

In another embodiment the invention relates to a pharmaceutical composition comprising a compound of formula (I) for use in a method for treatment of a patient with bacterial or fungal severe sepsis and bacterial or fungal septic shock and/or conditions arising therefrom, wherein the compound is selected from the group of compounds 1-95 shown in Table 1 below or a pharmaceutically acceptable salt thereof.

A pharmaceutical composition comprising a compound of formula (I) or a pharmaceutically acceptable salt thereof also comprises a pharmaceutically acceptable excipient, for use in a method for treatment of a patient with bacterial or fungal severe sepsis and septic shock and/or conditions arising therefrom.

Table 1 below shows specific compounds that can be used in pharmaceutical compositions described herein

TABLE 1
specific compounds for treating bacterial or fungal severe sepsis and septic shock and/or
conditions arising therefrom

| Cpd | | |
|---|---|---|
| No. | Structure | Compound Name |
| 1 | 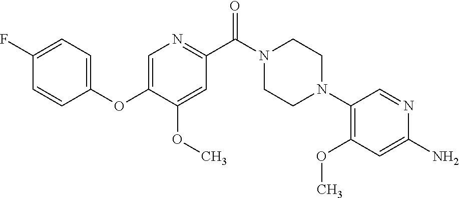 [SMILES: COc1cc(C(=O)N2CCN(c3cnc(N)cc3OC)CC2)ncc1Oc1ccc(F)cc1] | [4-(6-Amino-4-methoxy-pyridin-3-yl)- piperazin-1-yl]-[5-(4-fluoro-phenoxy)- 4-methoxy-pyridin-2-yl]-methanone |
| 2 | 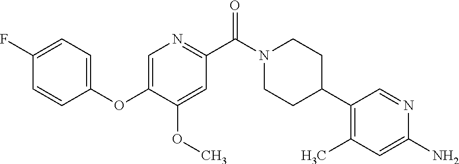 [SMILES: COc1cc(C(=O)N2CCC(c3cnc(N)cc3C)CC2)ncc1Oc1ccc(F)cc1] | (6-Amino-4-methyl-3′,4′,5′,6′- tetrahydro-2′H-[3,4′]bipyridinyl-1′-yl)- [5-(4-fluoro-phenoxy)-4-methoxy- pyridin-2-yl]-methanone |
| 3 | 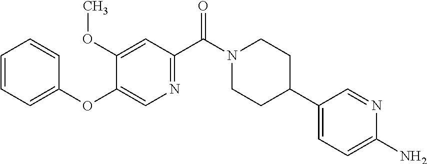 [SMILES: COc1cc(C(=O)N2CCC(c3ccc(N)nc3)CC2)ncc1Oc1ccccc1] | (6-Amino-3′,4′,5′,6′-tetrahydro-2′H- [3,4′]bipyridinyl-1′-yl)-(4-methoxy-5- phenoxy-pyridin-2-yl)-methanone |
| 4 | 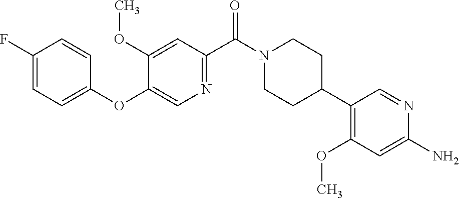 [SMILES: COc1cc(C(=O)N2CCC(c3cnc(N)cc3OC)CC2)ncc1Oc1ccc(F)cc1] | (6-Amino-4-methoxy-3′,4′,5′,6′- tetrahydro-2′H-[3,4′]bipyridinyl-1′-yl)- [5-(4-fluoro-phenoxy)-4-methoxy- pyridin-2-yl]-methanone |
| 5 | 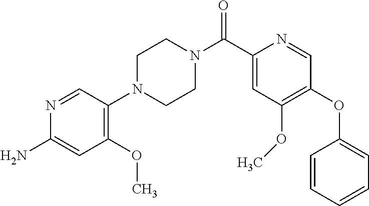 [SMILES: COc1cc(C(=O)N2CCN(c3cnc(N)cc3OC)CC2)ncc1Oc1ccccc1] | [4-(6-Amino-4-methoxy-pyridin-3-yl)- piperazin-1-yl]-(4-methoxy-5- phenoxy-pyridin-2-yl)-methanone |
| 6 | 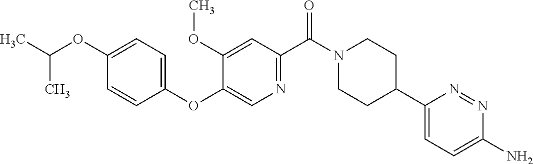 [SMILES: COc1cc(C(=O)N2CCC(c3ccc(N)nn3)CC2)ncc1Oc1ccc(OC(C)C)cc1] | [4-(6-Amino-pyridazin-3-yl)-piperidin- 1-yl]-[5-(4-isopropoxy-phenoxy)-4- methoxy-pyridin-2-yl]-methanone |
| 7 | 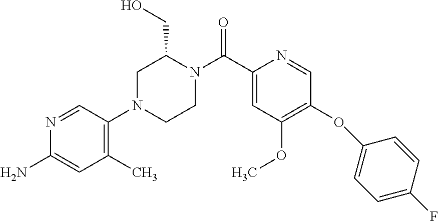 [SMILES: COc1cc(C(=O)N2CCN(c3cnc(N)cc3C)C[C@@H]2CO)ncc1Oc1ccc(F)cc1] | [(R)-4-(6-Amino-4-methyl-pyridin-3- yl)-2-hydroxymethyl-piperazin-1-yl]- [5-(4-fluoro-phenoxy)-4-methoxy- pyridin-2-yl]-methanone |
| 8 | 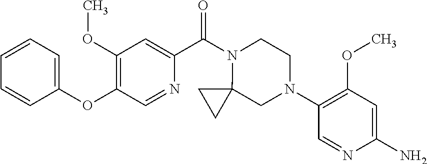 [SMILES: COc1cc(C(=O)N2CCN(c3cnc(N)cc3OC)CC23CC3)ncc1Oc1ccccc1] | [7-(6-Amino-4-methoxy-pyridin-3-yl)- 4,7-diaza-spiro[2.5]oct-4-yl]-(4- methoxy-5-phenoxy-pyridin-2-yl)- methanone |
| 9 | 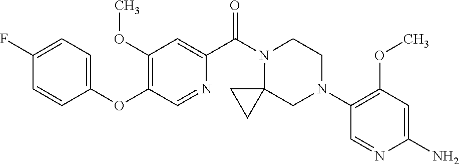 [SMILES: COc1cc(C(=O)N2CCN(c3cnc(N)cc3OC)CC23CC3)ncc1Oc1ccc(F)cc1] | [7-(6-Amino-4-methoxy-pyridin-3-yl)- 4,7-diaza-spiro[2.5]oct-4-yl]-[5-(4- fluoro-phenoxy)-4-methoxy-pyridin-2- yl]-methanone |
| 10 | 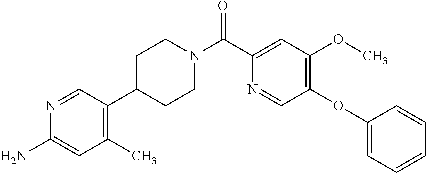 [SMILES: COc1cc(C(=O)N2CCC(c3cnc(N)cc3C)CC2)ncc1Oc1ccccc1] | (6-Amino-4-methyl-3′,4′,5′,6′- tetrahydro-2′H-[3,4′]bipyridinyl-1′-yl)- (4-methoxy-5-phenoxy-pyridin-2-yl)- methanone |
| 11 | 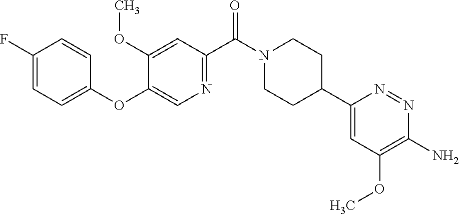 [SMILES: COc1cc(C(=O)N2CCC(c3cc(OC)c(N)nn3)CC2)ncc1Oc1ccc(F)cc1] | [4-(6-Amino-5-methoxy-pyridazin-3- yl)-piperidin-1-yl]-[5-(4-fluoro- phenoxy)-4-methoxy-pyridin-2-yl]- methanone |
| 12 | 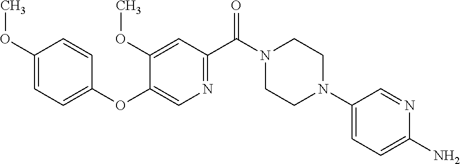 [SMILES: COc1ccc(Oc2cnc(C(=O)N3CCN(c4ccc(N)nc4)CC3)cc2OC)cc1] | [4-(6-Amino-pyridin-3-yl)-piperazin-1- yl]-[4-methoxy-5-(4-methoxy- phenoxy)-pyridin-2-yl]-methanone |
| 13 | 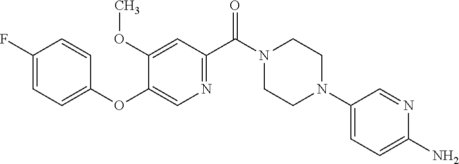 [SMILES: COc1cc(C(=O)N2CCN(c3ccc(N)nc3)CC2)ncc1Oc1ccc(F)cc1] | [4-(6-Amino-pyridin-3-yl)-piperazin-1- yl]-[5-(4-fluoro-phenoxy)-4-methoxy- pyridin-2-yl]-methanone |
| 14 | 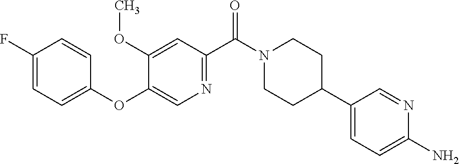 [SMILES: COc1cc(C(=O)N2CCC(c3ccc(N)nc3)CC2)ncc1Oc1ccc(F)cc1] | (6-Amino-3′,4′,5′,6′-tetrahydro-2′H- [3,4′]bipyridinyl-1′-yl)-[5-(4-fluoro- phenoxy)-4-methoxy-pyridin-2-yl]- methanone |
| 15 | 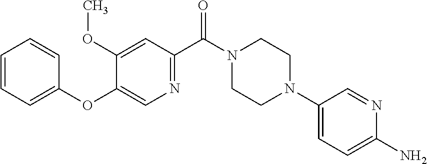 [SMILES: COc1cc(C(=O)N2CCN(c3ccc(N)nc3)CC2)ncc1Oc1ccccc1] | [4-(6-Amino-pyridin-3-yl)-piperazin-1- yl]-(4-methoxy-5-phenoxy-pyridin-2- yl)-methanone |
| 16 | 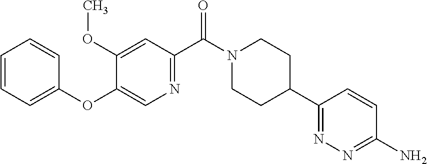 [SMILES: COc1cc(C(=O)N2CCC(c3ccc(N)nn3)CC2)ncc1Oc1ccccc1] | [4-(6-Amino-pyridazin-3-yl)-piperidin- 1-yl]-(4-methoxy-5-phenoxy-pyridin- 2-yl)-methanone |
| 17 | 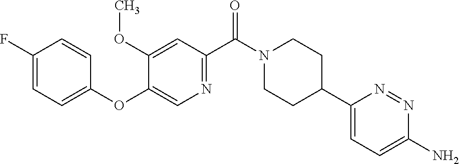 [SMILES: COc1cc(C(=O)N2CCC(c3ccc(N)nn3)CC2)ncc1Oc1ccc(F)cc1] | [4-(6-Amino-pyridazin-3-yl)-piperidin- 1-yl]-[5-(4-fluoro-phenoxy)-4- methoxy-pyridin-2-yl]-methanone |
| 18 | 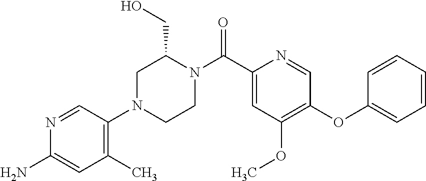 [SMILES: COc1cc(C(=O)N2CCN(c3cnc(N)cc3C)C[C@@H]2CO)ncc1Oc1ccccc1] | [(R)-4-(6-Amino-4-methyl-pyridin-3- yl)-2-hydroxymethyl-piperazin-1-yl]- (4-methoxy-5-phenoxy-pyridin-2-yl)- methanone |
| 19 | 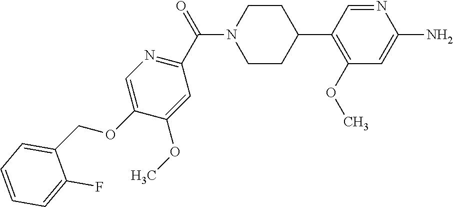 [SMILES: COc1cc(C(=O)N2CCC(c3cnc(N)cc3OC)CC2)ncc1OCc1ccccc1F] | (6-Amino-4-methoxy-3′,4′,5′,6′- tetrahydro-2′H-[3,4′]bipyridinyl-1′-yl)- [5-(2-fluoro-benzyloxy)-4-methoxy- pyridin-2-yl]-methanone |
| 20 | 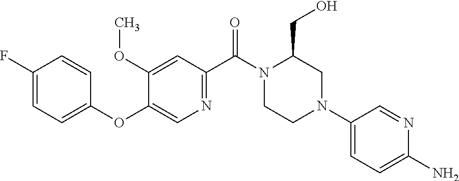 [SMILES: COc1cc(C(=O)N2CCN(c3ccc(N)nc3)C[C@@H]2CO)ncc1Oc1ccc(F)cc1] | [(R)-4-(6-Amino-pyridin-3-yl)-2- hydroxymethyl-piperazin-1-yl]-[5-(4- fluoro-phenoxy)-4-methoxy-pyridin-2- yl]-methanone |
| 21 | 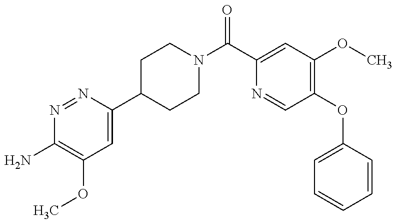 [SMILES: COc1cc(C(=O)N2CCC(c3cc(OC)c(N)nn3)CC2)ncc1Oc1ccccc1] | [4-(6-Amino-5-methoxy-pyridazin-3- yl)-piperidin-1-yl]-(4-methoxy-5- phenoxy-pyridin-2-yl)-methanone |
| 22 | 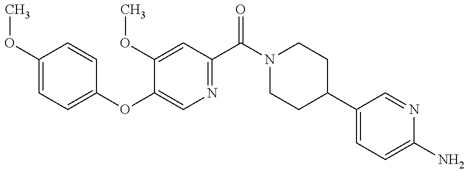 [SMILES: COc1ccc(Oc2cnc(C(=O)N3CCC(c4ccc(N)nc4)CC3)cc2OC)cc1] | (6-Amino-3′,4′,5′,6′-tetrahydro-2′H- [3,4′]bipyridinyl-1′-yl)-[4-methoxy-5- (4-methoxy-phenoxy)-pyridin-2-yl]- methanone |
| 23 | 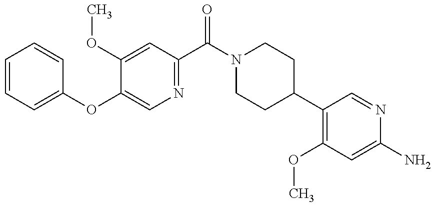 [SMILES: COc1cc(C(=O)N2CCC(c3cnc(N)cc3OC)CC2)ncc1Oc1ccccc1] | (6-Amino-4-methoxy-3′,4′,5′,6′- tetrahydro-2′H-[3,4′]bipyridinyl-1′-yl)- (4-methoxy-5-phenoxy-pyridin-2-yl)- methanone |
| 24 | 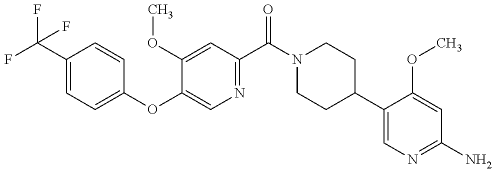 [SMILES: COc1cc(C(=O)N2CCC(c3cnc(N)cc3OC)CC2)ncc1Oc1ccc(C(F)(F)F)cc1] | (6-Amino-4-methoxy-3′,4′,5′,6′- tetrahydro-2′H-[3,4′]bipyridinyl-1′-yl)- [4-methoxy-5-(4-trifluoromethyl- phenoxy)-pyridin-2-yl]-methanone |
| 25 | 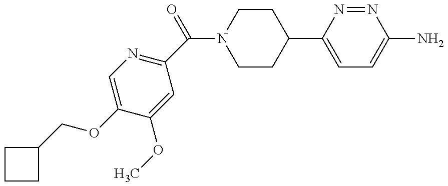 [SMILES: COc1cc(C(=O)N2CCC(c3ccc(N)nn3)CC2)ncc1OCC1CCC1] | [4-(6-Amino-pyridazin-3-yl)-piperidin- 1-yl]-(5-cyclobutylmethoxy-4- methoxy-pyridin-2-yl)-methanone |
| 26 | 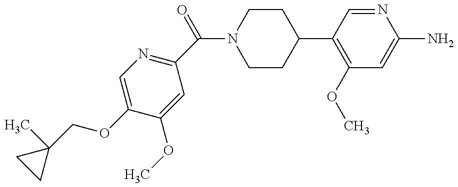 [SMILES: COc1cc(C(=O)N2CCC(c3cnc(N)cc3OC)CC2)ncc1OCC1(C)CC1] | (6-Amino-4-methoxy-3′,4′,5′,6′- tetrahydro-2′H-[3,4′]bipyridinyl-1′-yl)- [4-methoxy-5-(1-methyl- cyclopropylmethoxy)-pyridin-2-yl]- methanone |
| 27 | 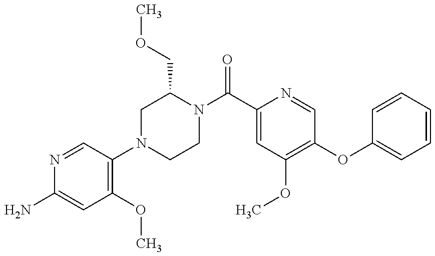 [SMILES: COC[C@H]1CN(c2cnc(N)cc2OC)CCN1C(=O)c1cc(OC)c(Oc2ccccc2)cn1] | [(R)-4-(6-Amino-4-methoxy-pyridin-3- yl)-2-methoxymethyl-piperazin-1-yl]- (4-methoxy-5-phenoxy-pyridin-2-yl)- methanone |
| 28 | 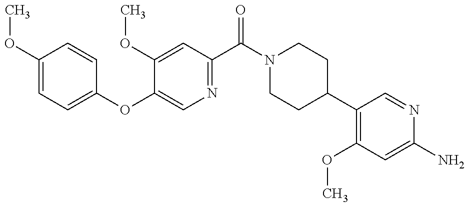 [SMILES: COc1ccc(Oc2cnc(C(=O)N3CCC(c4cnc(N)cc4OC)CC3)cc2OC)cc1] | (6-Amino-4-methoxy-3′,4′,5′,6′- tetrahydro-2′H-[3,4′]bipyridinyl-1′-yl)- [4-methoxy-5-(4-methoxy-phenoxy)- pyridin-2-yl]-methanone |
| 29 | 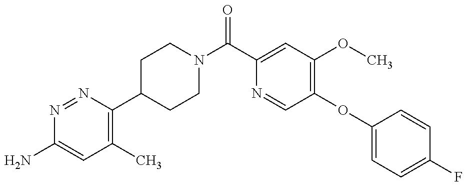 [SMILES: COc1cc(C(=O)N2CCC(c3nnc(N)cc3C)CC2)ncc1Oc1ccc(F)cc1] | [4-(6-Amino-4-methyl-pyridazin-3-yl)- piperidin-1-yl]-[5-(4-fluoro-phenoxy)- 4-methoxy-pyridin-2-yl]-methanone |
| 30 | 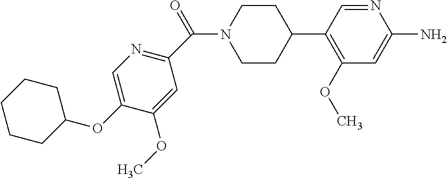 [SMILES: COc1cc(C(=O)N2CCC(c3cnc(N)cc3OC)CC2)ncc1OC1CCCCC1] | (6-Amino-4-methoxy-3′,4′,5′,6′- tetrahydro-2′H-[3,4′]bipyridinyl-1′-yl)- (5-cyclohexyloxy-4-methoxy-pyridin- 2-yl)-methanone |
| 31 | 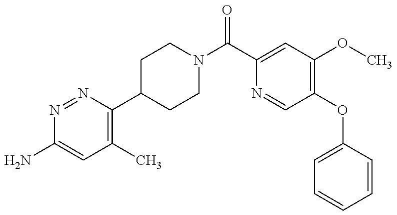 [SMILES: COc1cc(C(=O)N2CCC(c3nnc(N)cc3C)CC2)ncc1Oc1ccccc1] | [4-(6-Amino-4-methyl-pyridazin-3-yl)- piperidin-1-yl]-(4-methoxy-5- phenoxy-pyridin-2-yl)-methanone |
| 32 | 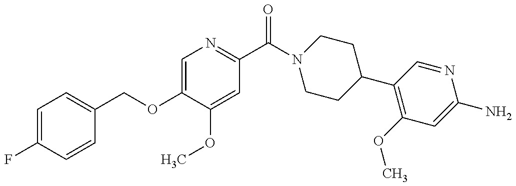 [SMILES: COc1cc(C(=O)N2CCC(c3cnc(N)cc3OC)CC2)ncc1OCc1ccc(F)cc1] | (6-Amino-4-methoxy-3′,4′,5′,6′- tetrahydro-2′H-[3,4′]bipyridinyl-1′-yl)- [5-(4-fluoro-benzyloxy)-4-methoxy- pyridin-2-yl]-methanone |
| 33 | 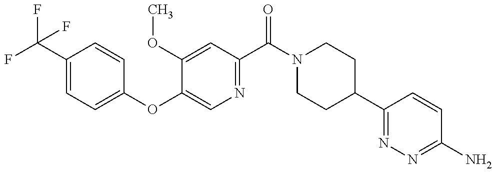 [SMILES: COc1cc(C(=O)N2CCC(c3ccc(N)nn3)CC2)ncc1Oc1ccc(C(F)(F)F)cc1] | [4-(6-Amino-pyridazin-3-yl)-piperidin- 1-yl]-[4-methoxy-5-(4-trifluoromethyl- phenoxy)-pyridin-2-yl]-methanone |
| 34 | 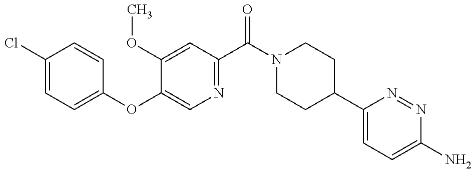 [SMILES: COc1cc(C(=O)N2CCC(c3ccc(N)nn3)CC2)ncc1Oc1ccc(Cl)cc1] | [4-(6-Amino-pyridazin-3-yl)-piperidin- 1-yl]-[5-(4-chloro-phenoxy)-4- methoxy-pyridin-2-yl]-methanone |
| 35 | 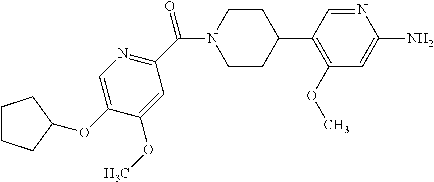 [SMILES: COc1cc(C(=O)N2CCC(c3cnc(N)cc3OC)CC2)ncc1OC1CCCC1] | (6-Amino-4-methoxy-3′,4′,5′,6′- tetrahydro-2′H-[3,4′]bipyridinyl-1′-yl)- (5-cyclopentyloxy-4-methoxy-pyridin- 2-yl)-methanone |
| 36 | 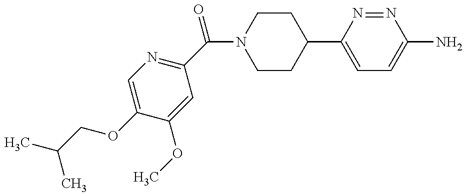 [SMILES: COc1cc(C(=O)N2CCC(c3ccc(N)nn3)CC2)ncc1OCC(C)C] | [4-(6-Amino-pyridazin-3-yl)-piperidin- 1-yl]-(5-isobutoxy-4-methoxy-pyridin- 2-yl)-methanone |
| 37 | 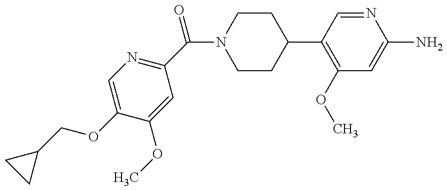 [SMILES: COc1cc(C(=O)N2CCC(c3cnc(N)cc3OC)CC2)ncc1OCC1CC1] | (6-Amino-4-methoxy-3′,4′,5′,6′- tetrahydro-2′H-[3,4′]bipyridinyl-1′-yl)- (5-cyclopropylmethoxy-4-methoxy- pyridin-2-yl)-methanone |
| 38 | 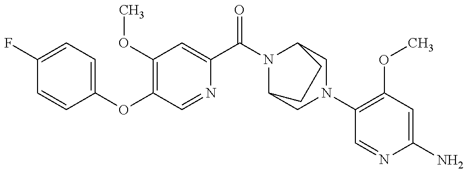 [SMILES: COc1cc(C(=O)N2C3CCC2CN(c2cnc(N)cc2OC)C3)ncc1Oc1ccc(F)cc1] | [3-(6-Amino-4-methoxy-pyridin-3-yl)- 3,8-diaza-bicyclo[3.2.1]oct-8-yl]-[5-(4- fluoro-phenoxy)-4-methoxy-pyridin-2- yl]-methanone |
| 39 | 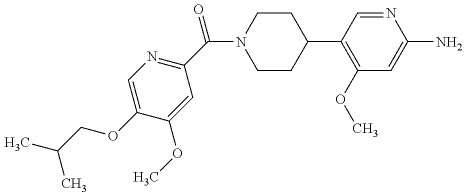 [SMILES: COc1cc(C(=O)N2CCC(c3cnc(N)cc3OC)CC2)ncc1OCC(C)C] | (6-Amino-4-methoxy-3′,4′,5′,6′- tetrahydro-2′H-[3,4′]bipyridinyl-1′-yl)- (5-isobutoxy-4-methoxy-pyridin-2-yl)- methanone |
| 40 | 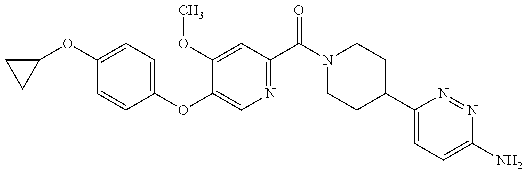 [SMILES: COc1cc(C(=O)N2CCC(c3ccc(N)nn3)CC2)ncc1Oc1ccc(OC2CC2)cc1] | [4-(6-Amino-pyridazin-3-yl)-piperidin- 1-yl]-[5-(4-cyclopropoxy-phenoxy)-4- methoxy-pyridin-2-yl]-methanone |
| 41 | 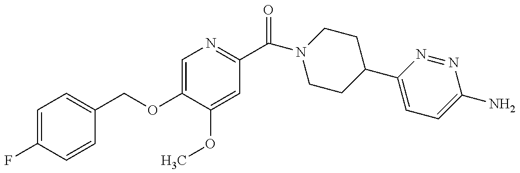 [SMILES: COc1cc(C(=O)N2CCC(c3ccc(N)nn3)CC2)ncc1OCc1ccc(F)cc1] | [4-(6-Amino-pyridazin-3-yl)-piperidin- 1-yl]-[5-(4-fluoro-benzyloxy)-4- methoxy-pyridin-2-yl]-methanone |
| 42 | 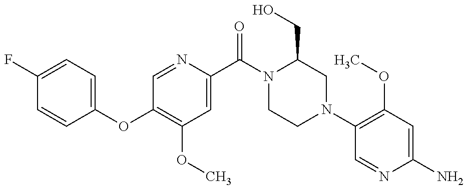 [SMILES: COc1cc(C(=O)N2CCN(c3cnc(N)cc3OC)C[C@@H]2CO)ncc1Oc1ccc(F)cc1] | [(R)-4-(6-Amino-4-methoxy-pyridin-3- yl)-2-hydroxymethyl-piperazin-1-yl]-[5- (4-fluoro-phenoxy)-4-methoxy-pyridin- 2-yl]-methanone |
| 43 | 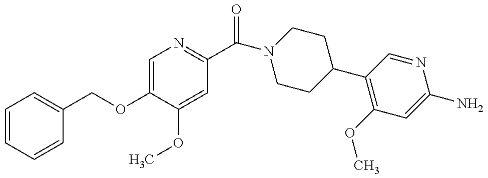 [SMILES: COc1cc(C(=O)N2CCC(c3cnc(N)cc3OC)CC2)ncc1OCc1ccccc1] | (6-Amino-4-methoxy-3′,4′,5′,6′- tetrahydro-2′H-[3,4′]bipyridinyl-1′-yl)- (5-benzyloxy-4-methoxy-pyridin-2-yl)- methanone |
| 44 | 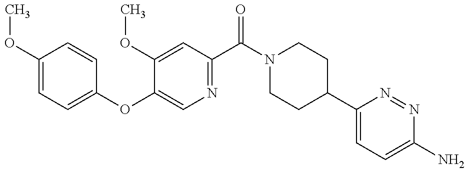 [SMILES: COc1ccc(Oc2cnc(C(=O)N3CCC(c4ccc(N)nn4)CC3)cc2OC)cc1] | [4-(6-Amino-pyridazin-3-yl)-piperidin- 1-yl]-[4-methoxy-5-(4-methoxy- phenoxy)-pyridin-2-yl]-methanone |
| 45 | 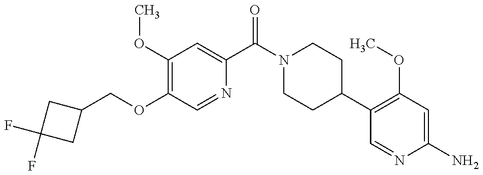 [SMILES: COc1cc(C(=O)N2CCC(c3cnc(N)cc3OC)CC2)ncc1OCC1CC(F)(F)C1] | (6-Amino-4-methoxy-3′,4′,5′,6′- tetrahydro-2′H-[3,4′]bipyridinyl-1′-yl)- [5-(3,3-difluoro-cyclobutylmethoxy)-4- methoxy-pyridin-2-yl]-methanone |
| 46 | 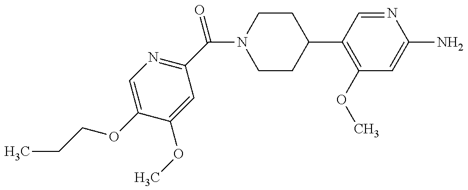 [SMILES: CCCOc1cnc(C(=O)N2CCC(c3cnc(N)cc3OC)CC2)cc1OC] | (6-Amino-4-methoxy-3′,4′,5′,6′- tetrahydro-2′H-[3,4′]bipyridinyl-1′-yl)- (4-methoxy-5-propoxy-pyridin-2-yl)- methanone |
| 47 | 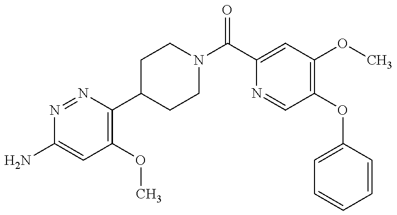 [SMILES: COc1cc(C(=O)N2CCC(c3nnc(N)cc3OC)CC2)ncc1Oc1ccccc1] | [4-(6-Amino-4-methoxy-pyridazin-3- yl)-piperidin-1-yl]-(4-methoxy-5- phenoxy-pyridin-2-yl)-methanone |
| 48 | 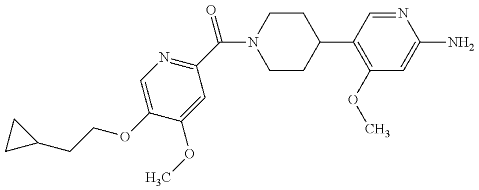 [SMILES: COc1cc(C(=O)N2CCC(c3cnc(N)cc3OC)CC2)ncc1OCCC1CC1] | (6-Amino-4-methoxy-3′,4′,5′,6′- tetrahydro-2′H-[3,4′]bipyridinyl-1′-yl)- [5-(2-cyclopropyl-ethoxy)-4-methoxy- pyridin-2-yl]-methanone |
| 49 | 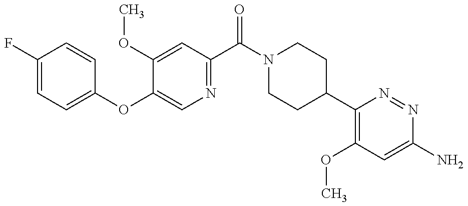 [SMILES: COc1cc(C(=O)N2CCC(c3nnc(N)cc3OC)CC2)ncc1Oc1ccc(F)cc1] | [4-(6-Amino-4-methoxy-pyridazin-3- yl)-piperidin-1-yl]-[5-(4-fluoro- phenoxy)-4-methoxy-pyridin-2-yl]- methanone |
| 50 | 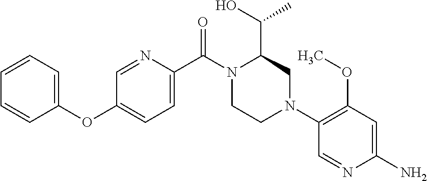 [SMILES: COc1cc(N)ncc1N1CCN(C(=O)c2ccc(Oc3ccccc3)cn2)[C@@H]([C@@H](C)O)C1] | (1R)-1-[(2R)-4-(6-amino-4- methoxypyridin-3-yl)-1-(5- phenoxypyridine-2-carbonyl)piperazin- 2-yl]ethan-1-ol |
| 51 | 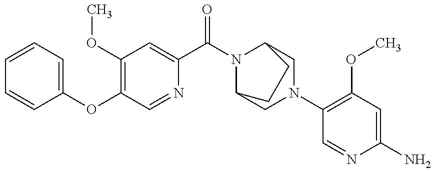 [SMILES: COc1cc(C(=O)N2C3CCC2CN(c2cnc(N)cc2OC)C3)ncc1Oc1ccccc1] | [3-(6-Amino-4-methoxy-pyridin-3-yl)- 3,8-diaza-bicyclo[3.2.1]oct-8-yl]-(4- methoxy-5-phenoxy-pyridin-2-yl)- methanone |
| 52 | 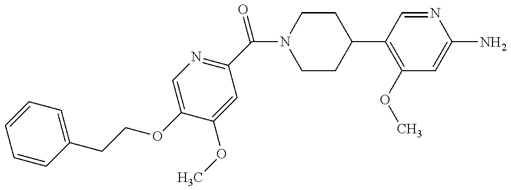 [SMILES: COc1cc(C(=O)N2CCC(c3cnc(N)cc3OC)CC2)ncc1OCCc1ccccc1] | (6-Amino-4-methoxy-3′,4′,5′,6′- tetrahydro-2′H-[3,4′]bipyridinyl-1′-yl)- (4-methoxy-5-phenethyloxy-pyridin-2- yl)-methanone |
| 53 | 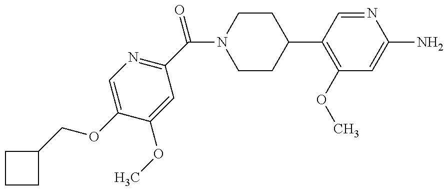 [SMILES: COc1cc(C(=O)N2CCC(c3cnc(N)cc3OC)CC2)ncc1OCC1CCC1] | (6-Amino-4-methoxy-3′,4′,5′,6′- tetrahydro-2′H-[3,4′]bipyridinyl-1′-yl)- (5-cyclobutylmethoxy-4-methoxy- pyridin-2-yl)-methanone |
| 54 | 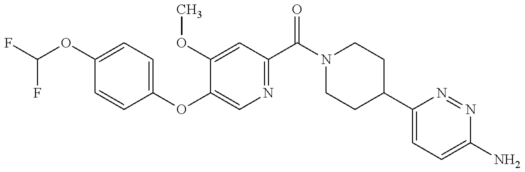 [SMILES: COc1cc(C(=O)N2CCC(c3ccc(N)nn3)CC2)ncc1Oc1ccc(OC(F)F)cc1] | [4-(6-Amino-pyridazin-3-yl)-piperidin- 1-yl]-[5-(4-difluoromethoxy-phenoxy)- 4-methoxy-pyridin-2-yl]-methanone |
| 55 | 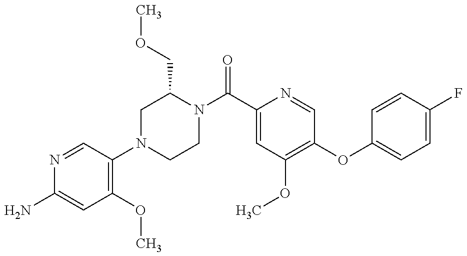 [SMILES: COC[C@H]1CN(c2cnc(N)cc2OC)CCN1C(=O)c1cc(OC)c(Oc2ccc(F)cc2)cn1] | [(R)-4-(6-Amino-4-methoxy-pyridin-3- yl)-2-methoxymethyl-piperazin-1-yl]- [5-(4-fluoro-phenoxy)-4-methoxy- pyridin-2-yl]-methanone |
| 56 | 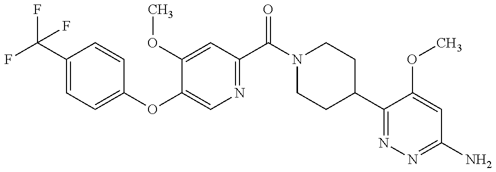 [SMILES: COc1cc(C(=O)N2CCC(c3nnc(N)cc3OC)CC2)ncc1Oc1ccc(C(F)(F)F)cc1] | [4-(6-Amino-4-methoxy-pyridazin-3- yl)-piperidin-1-yl]-[4-methoxy-5-(4- trifluoromethyl-phenoxy)-pyridin-2-yl]- methanone |
| 57 | 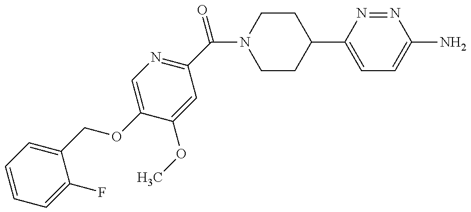 [SMILES: COc1cc(C(=O)N2CCC(c3ccc(N)nn3)CC2)ncc1OCc1ccccc1F] | [4-(6-Amino-pyridazin-3-yl)-piperidin- 1-yl]-[5-(2-fluoro-benzyloxy)-4- methoxy-pyridin-2-yl]-methanone |
| 58 | 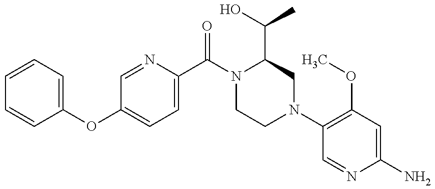 [SMILES: COc1cc(N)ncc1N1CCN(C(=O)c2ccc(Oc3ccccc3)cn2)[C@@H]([C@H](C)O)C1] | (1S)-1-[(2R)-4-(6-amino-4- methoxypyridin-3-yl)-1-(5- phenoxypyridine-2-carbonyl)piperazin- 2-yllethan-1-ol |
| 59 | 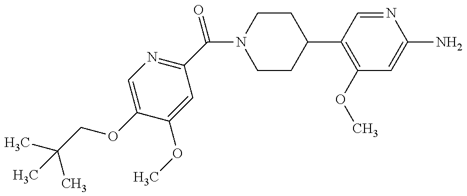 [SMILES: COc1cc(C(=O)N2CCC(c3cnc(N)cc3OC)CC2)ncc1OCC(C)(C)C] | (6-Amino-4-methoxy-3′,4′,5′,6′- tetrahydro-2′H-[3,4′]bipyridinyl-1′-yl)- [5-(2,2-dimethyl-propoxy)-4-methoxy- pyridin-2-yl]-methanone |
| 60 | 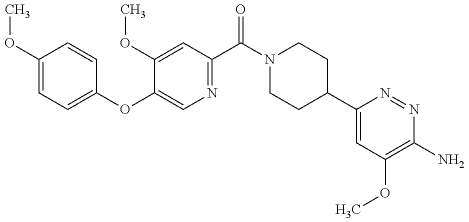 [SMILES: COc1ccc(Oc2cnc(C(=O)N3CCC(c4cc(OC)c(N)nn4)CC3)cc2OC)cc1] | [4-(6-Amino-5-methoxy-pyridazin-3- yl)-piperidin-1-yl]-[4-methoxy-5-(4- methoxy-phenoxy)-pyridin-2-yl]- methanone |
| 61 | 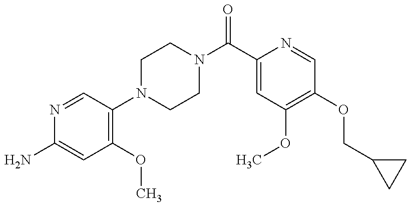 [SMILES: COc1cc(C(=O)N2CCN(c3cnc(N)cc3OC)CC2)ncc1OCC1CC1] | [4-(6-Amino-4-methoxy-pyridin-3-yl)- piperazin-1-yl]-(5-cyclopropylmethoxy- 4-methoxy-pyridin-2-yl)-methanone |
| 62 | 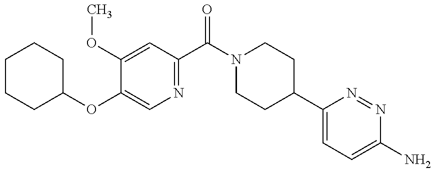 [SMILES: COc1cc(C(=O)N2CCC(c3ccc(N)nn3)CC2)ncc1OC1CCCCC1] | [4-(6-Amino-pyridazin-3-yl)-piperidin- 1-yl]-(5-cyclohexyloxy-4-methoxy- pyridin-2-yl)-methanone |
| 63 | 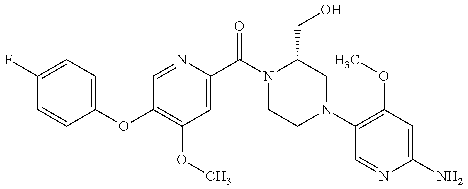 [SMILES: COc1cc(C(=O)N2CCN(c3cnc(N)cc3OC)C[C@H]2CO)ncc1Oc1ccc(F)cc1] | [(S)-4-(6-Amino-4-methoxy-pyridin-3- yl)-2-hydroxymethyl-piperazin-1-yl]-[5- (4-fluoro-phenoxy)-4-methoxy-pyridin- 2-yl]-methanone |
| 64 | 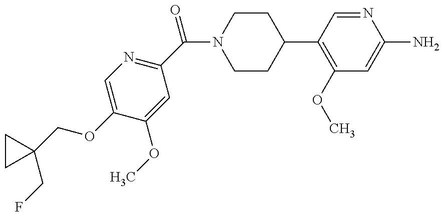 [SMILES: COc1cc(C(=O)N2CCC(c3cnc(N)cc3OC)CC2)ncc1OCC1(CF)CC1] | (6-Amino-4-methoxy-3′,4′,5′,6′- tetrahydro-2′H-[3,4′]bipyridinyl-1′-yl)- [5-(1-fluoromethyl- cyclopropylmethoxy)-4-methoxy- pyridin-2-yl]-methanone |
| 65 | 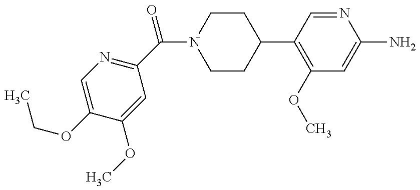 [SMILES: CCOc1cnc(C(=O)N2CCC(c3cnc(N)cc3OC)CC2)cc1OC] | (6-Amino-4-methoxy-3′,4′,5′,6′- tetrahydro-2′H-[3,4′]bipyridinyl-1′-yl)- (5-ethoxy-4-methoxy-pyridin-2-yl)- methanone |
| 66 | 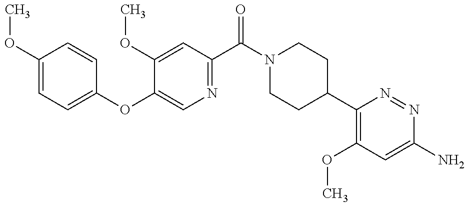 [SMILES: COc1ccc(Oc2cnc(C(=O)N3CCC(c4nnc(N)cc4OC)CC3)cc2OC)cc1] | [4-(6-Amino-4-methoxy-pyridazin-3- yl)-piperidin-1-yl]-[4-methoxy-5-(4- methoxy-phenoxy)-pyridin-2-yl]- methanone |
| 67 | 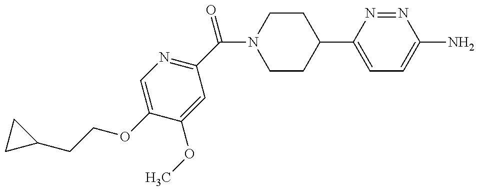 [SMILES: COc1cc(C(=O)N2CCC(c3ccc(N)nn3)CC2)ncc1OCCC1CC1] | [4-(6-Amino-pyridazin-3-yl)-piperidin- 1-yl]-[5-(2-cyclopropyl-ethoxy)-4- methoxy-pyridin-2-yl]-methanone |
| 68 | 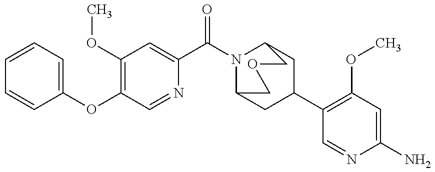 [SMILES: COc1cc(C(=O)N2C3COCC2CC(c2cnc(N)cc2OC)C3)ncc1Oc1ccccc1] | [7-(6-Amino-4-methoxy-pyridin-3-yl)-3- oxa-9-aza-bicyclo[3.3.1]non-9-yl]-(4- methoxy-5-phenoxy-pyridin-2-yl)- methanone |
| 69 | 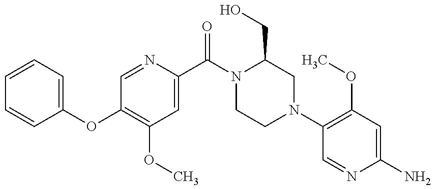 [SMILES: COc1cc(C(=O)N2CCN(c3cnc(N)cc3OC)C[C@@H]2CO)ncc1Oc1ccccc1] | [(R)-4-(6-Amino-4-methoxy-pyridin-3- yl)-2-hydroxymethyl-piperazin-1-yl]-(4- methoxy-5-phenoxy-pyridin-2-yl)- methanone |
| 70 | 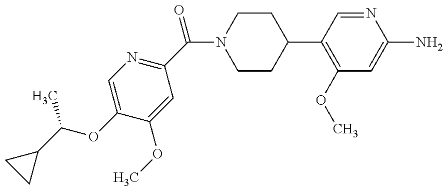 [SMILES: COc1cc(C(=O)N2CCC(c3cnc(N)cc3OC)CC2)ncc1O[C@@H](C)C1CC1] | (6-Amino-4-methoxy-3′,4′,5′,6′- tetrahydro-2′H-[3,4′]bipyridinyl-1′-yl)- [5-((S)-1-cyclopropyl-ethoxy)-4- methoxy-pyridin-2-yl]-methanone |
| 71 | 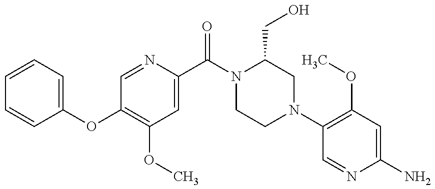 [SMILES: COc1cc(C(=O)N2CCN(c3cnc(N)cc3OC)C[C@H]2CO)ncc1Oc1ccccc1] | [(S)-4-(6-Amino-4-methoxy-pyridin-3- yl)-2-hydroxymethyl-piperazin-1-yl]-(4- methoxy-5-phenoxy-pyridin-2-yl)- methanone |
| 72 | 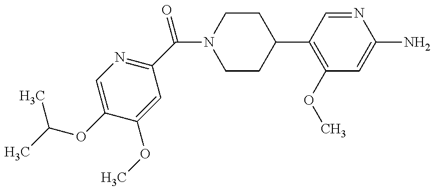 [SMILES: COc1cc(C(=O)N2CCC(c3cnc(N)cc3OC)CC2)ncc1OC(C)C] | (6-Amino-4-methoxy-3′,4′,5′,6′- tetrahydro-2′H-[3,4′]bipyridinyl-1′-yl)- (5-isopropoxy-4-methoxy-pyridin-2-yl)- methanone |
| 73 | 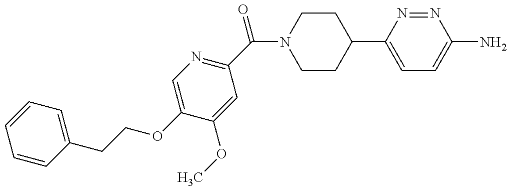 [SMILES: COc1cc(C(=O)N2CCC(c3ccc(N)nn3)CC2)ncc1OCCc1ccccc1] | [4-(6-Amino-pyridazin-3-yl)-piperidin- 1-yl]-(4-methoxy-5-phenethyloxy- pyridin-2-yl)-methanone |
| 74 | 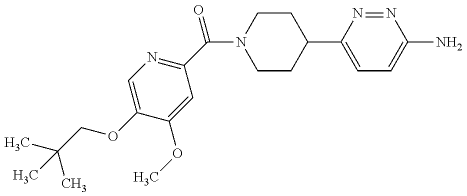 [SMILES: COc1cc(C(=O)N2CCC(c3ccc(N)nn3)CC2)ncc1OCC(C)(C)C] | [4-(6-Amino-pyridazin-3-yl)-piperidin- 1-yl]-[5-(2,2-dimethyl-propoxy)-4- methoxy-pyridin-2-yl]-methanone |
| 75 | 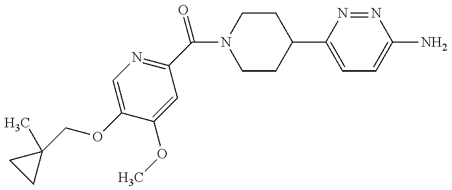 [SMILES: COc1cc(C(=O)N2CCC(c3ccc(N)nn3)CC2)ncc1OCC1(C)CC1] | [4-(6-Amino-pyridazin-3-yl)-piperidin- 1-yl]-[4-methoxy-5-(1-methyl- cyclopropylmethoxy)-pyridin-2-yl]- methanone |
| 76 | 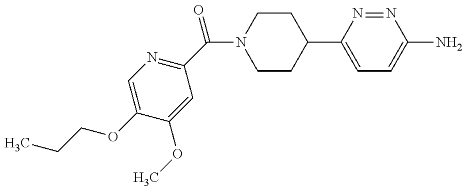 [SMILES: CCCOc1cnc(C(=O)N2CCC(c3ccc(N)nn3)CC2)cc1OC] | [4-(6-Amino-pyridazin-3-yl)-piperidin- 1-yl]-(4-methoxy-5-propoxy-pyridin-2- yl)-methanone |
| 77 | 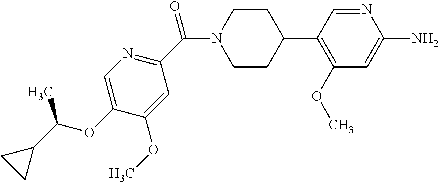 [SMILES: COc1cc(C(=O)N2CCC(c3cnc(N)cc3OC)CC2)ncc1O[C@H](C)C1CC1] | (6-Amino-4-methoxy-3′,4′,5′,6′- tetrahydro-2′H-[3,4′]bipyridinyl-1′-yl)- [5-((R)-1-cyclopropyl-ethoxy)-4- methoxy-pyridin-2-yl]-methanone |
| 78 | 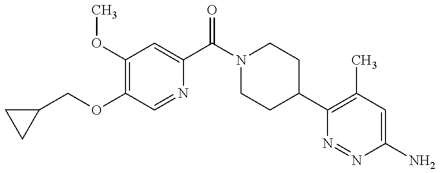 [SMILES: COc1cc(C(=O)N2CCC(c3nnc(N)cc3C)CC2)ncc1OCC1CC1] | [4-(6-Amino-4-methyl-pyridazin-3-yl)- piperidin-1-yl]-(5-cyclopropylmethoxy- 4-methoxy-pyridin-2-yl)-methanone |
| 79 | 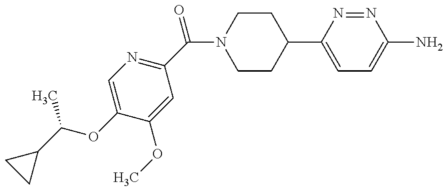 [SMILES: COc1cc(C(=O)N2CCC(c3ccc(N)nn3)CC2)ncc1O[C@@H](C)C1CC1] | [4-(6-Amino-pyridazin-3-yl)-piperidin- 1-yl]-[5-((S)-1-cyclopropyl-ethoxy)-4- methoxy-pyridin-2-yl]-methanone |
| 80 | 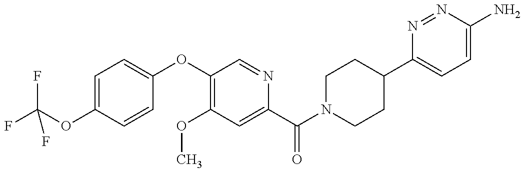 [SMILES: COc1cc(C(=O)N2CCC(c3ccc(N)nn3)CC2)ncc1Oc1ccc(OC(F)(F)F)cc1] | [4-(6-Amino-pyridazin-3-yl)-piperidin- 1-yl]-[4-methoxy-5-(4-trifluoromethoxy- phenoxy)-pyridin-2-yl]-methanone |
| 81 | 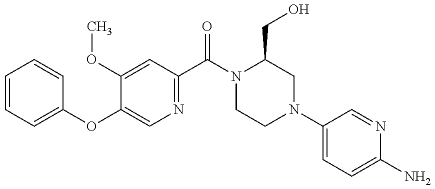 [SMILES: COc1cc(C(=O)N2CCN(c3ccc(N)nc3)C[C@@H]2CO)ncc1Oc1ccccc1] | [(R)-4-(6-Amino-pyridin-3-yl)-2- hydroxymethyl-piperazin-1-yl]-(4- methoxy-5-phenoxy-pyridin-2-yl)- methanone |
| 82 | 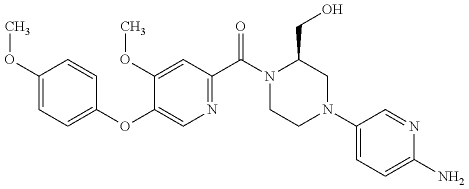 [SMILES: COc1ccc(Oc2cnc(C(=O)N3CCN(c4ccc(N)nc4)C[C@@H]3CO)cc2OC)cc1] | [(R)-4-(6-Amino-pyridin-3-yl)-2- hydroxymethyl-piperazin-1-yl]-[4- methoxy-5-(4-methoxy-phenoxy)- pyridin-2-yl]-methanone |
| 83 | 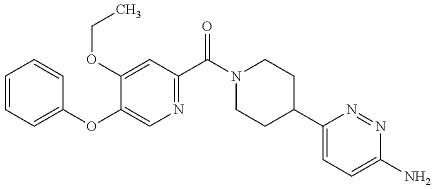 [SMILES: CCOc1cc(C(=O)N2CCC(c3ccc(N)nn3)CC2)ncc1Oc1ccccc1] | [4-(6-Amino-pyridazin-3-yl)-piperidin- 1-yl]-[5-(phenoxy)-4-ethoxy-pyridin-2- yl]-methanone |
| 84 | 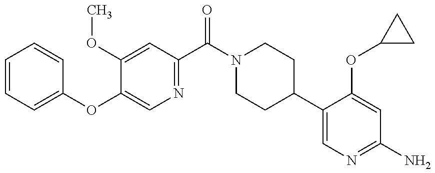 [SMILES: COc1cc(C(=O)N2CCC(c3cnc(N)cc3OC3CC3)CC2)ncc1Oc1ccccc1] | (6-Amino-4-cyclopropoxy-3′,4′,5′,6′- tetrahydro-2′H-[3,4′]bipyridinyl-1′-yl)- [5-(phenoxy)-4-methoxy-pyridin-2-yl]- methanone |
| 85 | 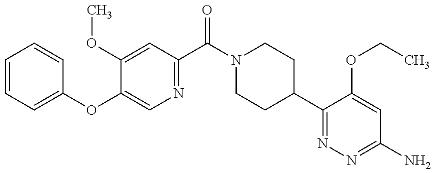 [SMILES: CCOc1cc(N)nnc1C1CCN(C(=O)c2cc(OC)c(Oc3ccccc3)cn2)CC1] | [4-(6-Amino-4-ethoxy-pyridazin-3-yl)- piperidin-1-yl]-[4-methoxy-5- (phenoxy)-pyridin-2-yl]-methanone |
| 86 | 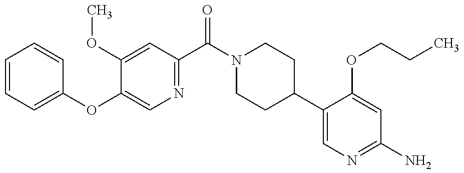 [SMILES: CCCOc1cc(N)ncc1C1CCN(C(=O)c2cc(OC)c(Oc3ccccc3)cn2)CC1] | (6-Amino-4-propoxy-3′,4′,5′,6′- tetrahydro-2′H-[3,4′]bipyridinyl-1′-yl)- [5-(phenoxy)-4-methoxy-pyridin-2-yl]- methanone |
| 87 | 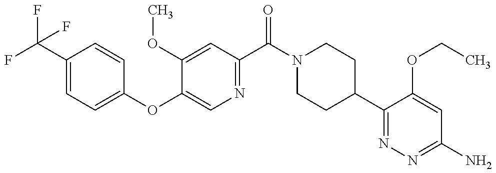 [SMILES: CCOc1cc(N)nnc1C1CCN(C(=O)c2cc(OC)c(Oc3ccc(C(F)(F)F)cc3)cn2)CC1] | 5-Ethoxy-6-(1-{4-methoxy-5-[4- (trifluoromethyl)phenoxy]pyridine-2- carbonyl}piperidin-4-yl)pyridazin-3- amine |
| 88 | 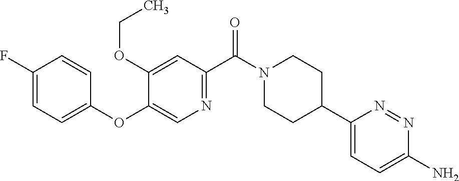 [SMILES: CCOc1cc(C(=O)N2CCC(c3ccc(N)nn3)CC2)ncc1Oc1ccc(F)cc1] | [4-(6-Amino-pyridazin-3-yl)-piperidin- 1-yl]-[5-(4-fluoro-phenoxy)-4-ethoxy- pyridin-2-yl]-methanone |
| 89 | 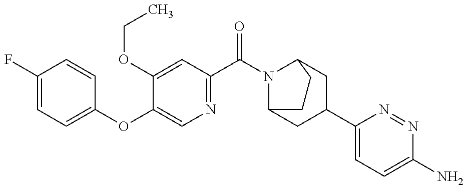 [SMILES: CCOc1cc(C(=O)N2C3CCC2CC(c2ccc(N)nn2)C3)ncc1Oc1ccc(F)cc1] | [3-(6-Amino-pyridazin-3-yl)-8-aza- bicyclo[3.2.1]oct-8-yl]-[4-ethoxy-5-(4- fluoro-phenoxy)-pyridin-2-yl]- methanone |
| 90 | 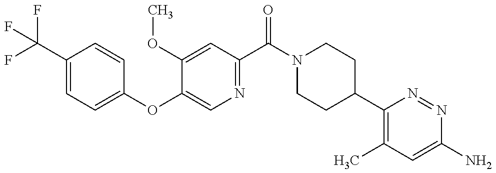 [SMILES: COc1cc(C(=O)N2CCC(c3nnc(N)cc3C)CC2)ncc1Oc1ccc(C(F)(F)F)cc1] | 6-(1-{4-Methoxy-5-[4-(trifluoro- methyl)phenoxy]pyridine-2- carbonyl}piperidin-4-yl)-5- methylpyridazin-3-amine |
| 91 | 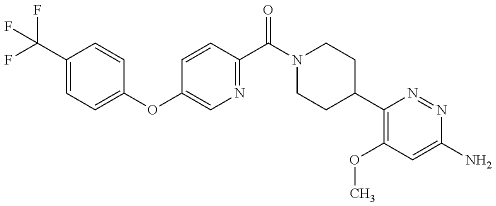 [SMILES: COc1cc(N)nnc1C1CCN(C(=O)c2ccc(Oc3ccc(C(F)(F)F)cc3)cn2)CC1] | 5-Methoxy-6-(1-{5-[4-(trifluoromethyl)- phenoxy]-pyridine-2- carbonyl}piperidin-4-yl)-pyridazin-3- amine |
| 92 | 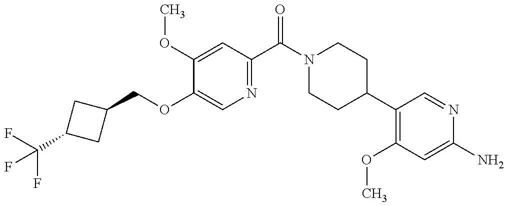 [SMILES: COc1cc(C(=O)N2CCC(c3cnc(N)cc3OC)CC2)ncc1OC[C@H]1C[C@H](C(F)(F)F)C1] | 4-Methoxy-5-[1-(4-methoxy-5-{[trans- 3-(trifluoromethyl)cyclobutyl]- methoxy}pyridine-2-carbonyl)- piperidin-4-yl]pyridin-2-amine |
| 93 | 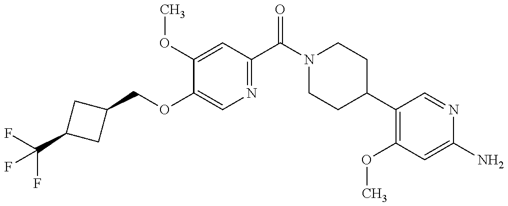 [SMILES: COc1cc(C(=O)N2CCC(c3cnc(N)cc3OC)CC2)ncc1OC[C@H]1C[C@@H](C(F)(F)F)C1] | 4-Methoxy-5-[1-(4-methoxy-5-{[(cis-3- (trifluoromethyl)-cyclobutyl]methoxy}- pyridine-2-carbonyl)piperidin-4- yllpyridin-2-amine |
| 94 | 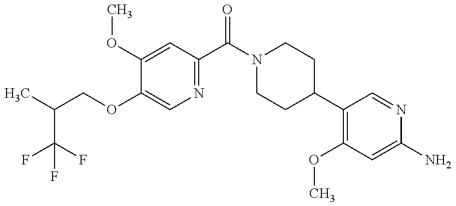 [SMILES: COc1cc(C(=O)N2CCC(c3cnc(N)cc3OC)CC2)ncc1OCC(C)C(F)(F)F] | 4-Methoxy-5-(1-{4-methoxy-5-[(2)- 3,3,3-trifluoro-2-methylpropoxy]- pyridine-2-carbonyl}piperidin-4- yl)pyridin-2-amine |
| 95 | 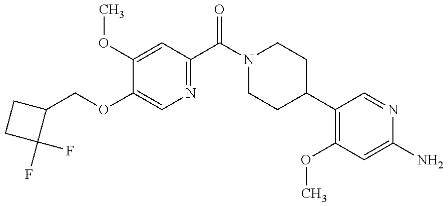 [SMILES: COc1cc(C(=O)N2CCC(c3cnc(N)cc3OC)CC2)ncc1OCC1CCC1(F)F] | 5-(1-{5-[(2,2-Difluorocyclobutyl)- methoxy]-4-methoxy-pyridine-2- carbonyl}-piperidin-4-yl)-4- methoxypyridin-2-amine |

FIGS. 2.1 to 2.4 of Example 2 and FIGS. 3.1 to 3.3 of Example 3 show that compound 17 significantly reduces vascular leakage, similar to compound 2 of WO/2029/161010.

### BRIEF DESCRIPTION OF THE FIGURES

In several of the figures, the following symbols are used to denote error bars and/or the statistical significance of the results:
The symbol “T” at the top of the bar graphs denotes the upper error bar (the lower error bar is not shown).
The symbol “*” denotes the statistical significance at p<0.05 between the positive control (CLP group or LPS group) and treated groups with the TRPC6 inhibitor.
The symbol “#” denotes the statistical significance at p<0.05 between the sham or PBS groups and the positive control (CLP or LPS).

FIG. 1.1 shows the effect of compound 2 of WO 2019/161010 in the mouse CLP model in Lung-dry tissue.

FIG. 1.2 shows the effect of compound 2 of WO 2019/161010 in the mouse CLP model in Liver-dry tissue.

FIG. 1.3 shows the effect of compound 2 of WO 2019/161010 in the mouse CLP model in Kidney-dry tissue.

FIG. 1.4 shows the effect of compound 2 of WO 2019/161010 in the mouse CLP model in Hearr-dry tissue.

FIG. 2.1 shows the effect of compound 17 in the mouse CLP model in Lung-dry tissue.

FIG. 2.2 shows the effect of compound 17 in the mouse CLP model in Liver-dry tissue.

FIG. 2.3 shows the effect of compound 17 in the mouse CLP model in Kidney-dry tissue.

FIG. 2.4 shows the effect of compound 17 in the mouse CLP model in Heart-dry tissue.

FIG. 3.1 shows the amount of total protein in BALF after LPS treatment without or after pretreatment with (+) compound 17.

FIG. 3.2 shows the amount of total protein in BALF after LPS treatment without or after pretreatment with (+) compound 17.

### DETAILED DESCRIPTION OF THE INVENTION

The compounds shown in Table 1 may be prepared according to procedures described in WO2019/081637 and WO 2019/161010.

The invention relates to a pharmaceutical composition comprising any of the compounds 1 to 95 depicted in Table 1, and the pharmaceutically acceptable salts thereof, for use in a method for treatment of a patient with a systemic inflammatory response to a bacterial or fungal infection such as sepsis, bacterial or fungal severe sepsis, or bacterial or fungal septic shock, or a condition arising therefrom which is selected from the group consisting of ARDS, related to infection, severe acute respiratory syndrome (SARS), and middle eastern respiratory syndrome (MERS).

In a particular embodiment any one of compounds 6, 16, 17, 29, 31, 33, 34, 40, 41, 44, 49, 54, 56, 57, 66, 80, 83, 85, 87, 88 and 90 depicted in Table 1, or a pharmaceutically acceptable salt thereof, is comprised by the pharmaceutical composition administered to a patient.

A pharmaceutical composition comprising any compound for use in the treatment with one or more of the compounds 1 to 95 of Table 1 disclosed hereinbefore is understood to have a corresponding embodiment in the European second medical use format “pharmaceutical composition comprising compound X for use in the therapy of disease Y”,
wherein compound X stands for a compound of formula (I) or one or more of the compounds 1 to 95 disclosed hereinbefore, and disease Y stands for a disorder associated with bacterial or fungal sepsis, bacterial or fungal severe sepsis, or bacterial or fungal septic shock.

In another embodiment the invention relates to a pharmaceutical composition comprising a compound of formula (I), as defined hereinbefore, for use in the treatment of bacterial or fungal severe sepsis and septic shock and/or conditions arising therefrom.

### GENERAL DEFINITIONS

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkyl” means an aryl group, which is bound to a C₁₋₃-alkyl-group, the latter of which is bound to the core or to the group to which the substituent is attached.

In case a compound is depicted in form of a chemical name and as a formula in case of any discrepancy the formula shall prevail.

The term “substituted” as used herein, means that any one or more hydrogens on the designated atom is replaced with a selection from the indicated group, provided that the designated atom's normal valence is not exceeded, and that the substitution results in a stable compound.

Unless specifically indicated, throughout the specification and the appended claims, a given chemical formula or name shall encompass tautomers and all stereo, optical and geometrical isomers (e.g. enantiomers, diastereomers, E/Z isomers etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include acetates, ascorbates, benzenesulfonates, benzoates, besylates, bicarbonates, bitartrates, bromides/hydrobromides, edetates, camsylates, carbonates, chlorides/hydrochlorides, citrates, edisylates, ethane disulfonates, estolates esylates, formates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates, subacetates, succinates, sulfamides, sulfates, tannates, tartrates, teoclates, toluenesulfonates, triethiodides, trifluoroacetates, ammonium, benzathines, chloroprocaines, cholines, diethanolamines, ethylenediamines, meglumines and procaines. Further pharmaceutically acceptable salts can be formed with cations from metals like aluminium, calcium, lithium, magnesium, potassium, sodium, zinc and the like (also see Pharmaceutical salts, Birge, S. M. et al., J. Pharm. Sci., (1977), 66, 1-19) or with cations from ammonia, L-arginine, calcium, 2,2′-iminobisethanol, L-lysine, magnesium, N-methyl-D-glucamine, potassium, sodium and tris(hydroxymethyl)-aminomethane.

The term halogen generally denotes fluorine, chlorine, bromine and iodine.

The term “C₁₋ₙ-alkyl”, wherein n is an integer selected from the group consisting of 2, 3, 4, 5 or 6, preferably 4 or 6, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₙ-cycloalkyl”, wherein n is an integer from 4 to n, either alone or in combination with another radical denotes a cyclic, saturated, unbranched hydrocarbon radical with 3 to n C atoms. For example, the term C₃₋₇-cycloalkyl includes cyclopropyl, cyclobutyl, cyclopentyl, cyclohexyl and cycloheptyl.

By the term “halo” added to an “alkyl”, “alkylene” or “cycloalkyl” group (saturated or unsaturated) is such an alkyl or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—. Analogously, the term “halo” added to an aryl group (e.g., phenyl) means that one or more hydrogen atoms are replaced by a halogen atom selected from among fluorine, chlorine or bromine, preferably fluorine and chlorine, particularly preferred is fluorine.

The term “carbocyclyl” as used either alone or in combination with another radical, means a mono- bi- or tricyclic ring structure consisting of 3 to 9 carbon atoms and optionally a heteroatom selected from the group consisting of N, O, and S. The term “carbocyclyl” refers to fully saturated ring systems and encompasses fused, bridged and spirocyclic systems.

Many of the terms given above may be used repeatedly in the definition of a formula or group and in each case have one of the meanings given above, independently of one another.

Unless specifically indicated, throughout the specification and the appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

Some of the compounds in Table 1 can exist in more than one tautomeric form. The invention includes methods for using all such tautomers.

In addition, within the scope of the invention is the use of prodrugs of the compounds of formula (I) for use in a method for treatment of a patient with bacterial or fungal severe sepsis and septic shock. Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

For all compounds disclosed herein above in this application, in the event the nomenclature is in conflict with the structure, it shall be understood that the compound is defined by the structure.

### Methods of Therapeutic Use

The compounds disclosed herein are particularly effective for treating bacterial and fungal severe sepsis and septic shock.

In one embodiment, the present invention provides compounds of formula (I) for use in a method for treatment of a patient with bacterial and fungal severe sepsis and septic shock by administering to the patient in need thereof a pharmaceutically effective amount of the compound of formula (I) as defined hereinbefore or of a compound selected from compounds 1 to 95 of Table 1, but preferably of a compound selected from compounds 6, 16, 17, 29, 31, 33, 34, 40, 41, 44, 49, 54, 56, 57, 66, 80, 83, 85, 87, 88 and 90 depicted in Table 1, or a pharmaceutically acceptable salt thereof.

In the context of treating sepsis, the most common cause of the Acute Respiratory Distress Syndrome (ARDS), lung inflammation characterized by an increase in lung vascular permeability and/or lung edema, the present invention also provides compounds of formula (I) for use in methods for treatment of a patient with ARDS by administering to the patient in need thereof a pharmaceutically effective amount of the compound of formula (I) as defined hereinbefore or of a compound selected from the group consisting of compounds 1 to 95, but preferably of a compound selected from the group consisting of compounds 6, 16, 17, 29, 31, 33, 34, 40, 41, 44, 49, 54, 56, 57, 66, 80, 83, 85, 87, 88 and 90 depicted in Table 1, or a pharmaceutically acceptable salt thereof.

In another embodiment, the present invention relates to the treatment of a respiratory disorder or condition arising from a bacterial infection, wherein the respiratory disorder or condition is selected from the group consisting of lung vascular hyperpermeability, pulmonary (lung) edema, lung ischemia reperfusion, acute respiratory distress syndrome (ARDS), acute lung injury (ALI), and severe acute respiratory syndrome (SARS) by administering to a patient in need thereof a pharmaceutically effective amount of a compound of formula (I) as defined hereinbefore or of a compound selected from the group consisting of compounds 1 to 95, but preferably of a compound selected from compounds 6, 16, 17, 29, 31, 33, 34, 40, 41, 44, 49, 54, 56, 57, 66, 80, 83, 85, 87, 88 and 90 depicted in Table 1, or a pharmaceutically acceptable salt thereof.

For use in the treatment of bacterial or fungal severe sepsis and bacterial or fungal septic shock the compounds of the invention may be administered via a pharmaceutical composition in any conventional pharmaceutical dosage form in any conventional manner such as a composition comprising a therapeutically effective amount of a compound according to the invention in the range from 0.1 to 90 wt.-% of the composition as a whole, preferably in the range from 0.5 to 50 wt.-% of the composition as a whole, or a pharmaceutically acceptable salt thereof. Conventional dosage forms typically include a pharmaceutically acceptable carrier suitable to the particular dosage form selected. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the compounds of formula (I), facilitate administration of pharmaceutical compositions containing them in certain embodiments, provide increased dissolution or dispersion, increase inhibitory activity, provide adjunct therapy, and the like, including other active ingredients. In one embodiment, for example, multiple compounds of the present invention can be administered. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of the invention (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either serially or in parallel). Separate dosing allows for greater flexibility in the dosing regimen.

As mentioned above, dosage forms of the compounds of this invention may include pharmaceutically acceptable carriers and adjuvants known to those of ordinary skill in the art and suitable to the dosage form. These carriers and adjuvants include, for example, ion exchangers, alumina, aluminum stearate, lecithin, serum proteins, buffer substances, water, salts or electrolytes and cellulose-based substances. Preferred dosage forms include tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements for the compounds of the present invention may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The compounds of the invention may be used alone or in combination of one or more additional therapeutic agents. Nonlimiting examples of additional therapeutic agents may include:
antimalarials such as hydroxychloroquine or chloroquine, each with or without azithromycin,
angiotensin II receptor antagonists (angiotensin receptor blockers (ARBs)) such as candesartan, eprosartan, candesartan, irbesartan, losartan, olmesartan, telmisartan, valsartan, azilsartan, and olmesartan medoxomil,
angiotensin converting enzyme inhibitors (e.g., benazepril, captopril, enalapril, fosinopril, lisinopril, moexipril, and perindopril),
anticoagulants (e.g. dabigatran, actylise, Warfarin, heparin, and acetylsalicylic acid),
antidiabetics such as alpha-glucosidase inhibitors (e.g., miglitol and acarbose), amylin analogs (e.g., pramlintide), dipeptidyl peptidase 4 inhibitors (e.g., alogliptin, sitagliptin, saxagliptin, and linagliptin), incretin mimetics (e.g., liraglutide, exenatide, liraglutide, exenatide, dulaglutide, albiglutide, and lixisenatide), insulin, meglitinides (e.g., repaglinide and nateglinide), biguanides (e.g., metformin); SGLT-2 inhibitors (e.g., canagliflozin, empagliflozin, and dapagliflozin), sulfonylureas (e.g., chlorpropamide, glimepiride, glyburide, glipizide, glyburide, tolazamide, and tolbutamide), and
thiazolidinediones (e.g., rosiglitazone and pioglitazone); CGRP antagonists (such as olcegepant, vaczegepant or zavegepant),
bronchodilators including short-acting and long-action beta agonists (e.g., albuterol, levalbuterol, salmeterol, formoterol, arformoterol, vilanterol, indacaterol and olodaterol) and short- and long-acting anticholinergics (ipratropium, tiotropium, umeclidinium, glycopyrrolatei and aclidinium),
steroids such as fluticasone and budesonide; and
corticosteroids such as dexamethasone (Dex), prednisone, methylprednisolone, and hydrocortisonea.

In yet another embodiment the compounds of the invention may be used in combination with various kinase inhibitors providing immunomodulatory effects (A. P. Kater et al., Blood Adv. 2021 Feb. 9; 5(3): 913-925), such as TKIs approved or in late-stage development for the treatment of hematological malignancies.

In a further embodiment the compounds of the invention may be used in combination with antifibrotics, such as nintedanib or pirfenidone, as patients in need of mechanical ventilation tend to develop lung fibrosis.

When used as combination treatment of a pharmaceutical combination, the compounds of the invention and the one or more additional agents can be administered in the same dosage form or different dosage forms. The compounds of the invention and the one or more additional agents can be administered simultaneously or separately, as part of a regimen.

It has been found that compounds of formula (I), wherein the groups R1 to R7 have the meanings given above, surprisingly can be used to treat a patient with a systemic response to bacteria, fungi or circulating bacterial or fungal products (bacterial or fungal sepsis). Thus, the compounds according to the invention may be used for the treatment of bacterial or fungal severe sepsis.

### EXAMPLES

### CLP-Induced Polymicrobial Sepsis in the Mouse

Cecal ligation puncture (CLP) is a model of polymicrobial sepsis which consists of extruding fecal content in the abdominal cavity of the animals under anesthesia.

Two models were performed:
Acute model of CLP lasting 24 h to measure vascular hyper-permeability in lung, liver, kidney and heart tissue. The vascular hyper-permeability was followed by the extravasation of Evans blue injected intravenously and diffused and accumulated into the tissue. The vascular hyper-permeability was expressed as μg of Evans blue in 100 mg of dry tissue.
Chronic model of CLP lasting 8 days. CLP is performed on day 0 and the survival rate is followed for 8 consecutive days.

### Acute Model of CLP: Vascular Hyper-Permeability

Mice were anesthetized with ketamine (80 mg kg⁻¹, i.p.) and xylazine (10 mg kg⁻¹, i.p.). A 1-1.5 cm abdominal midline incision was made, and the caecum was located and tightly ligated at half the distance between distal pole and the base of the caecum with 4-0 silk suture (mild grade). The caecum was punctured through-and-through once with a 21-gauge needle after medium ligation. A small amount of stool was extruded to ensure that the wounds were patent. Then the cecum was replaced in its original position within the abdomen, which was closed with sutures in layers. For negative control animals, a sham surgery was performed: sham-operated animals underwent identical laparotomy but without cecal ligation or puncture. The animals were resuscitated immediately after surgery with 1 mL subcutaneous normal saline and returned to their cages. Experiment was terminated at 24 hrs after CLP.

### Example 1: Effect of Compound 2 of WO 2019/161010 on Acute Model of CLP

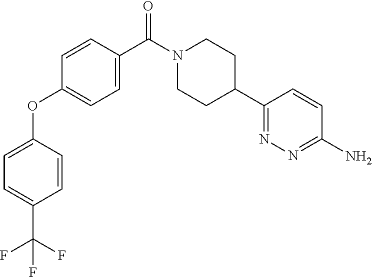 [SMILES: Nc1ccc(C2CCN(C(=O)c3ccc(Oc4ccc(C(F)(F)F)cc4)cc3)CC2)nn1]

Compound 2 of WO 2019/161010 or its vehicle (0.5% natrosol 0.010% Tween 80) was given orally (5 ml/kg) either prophylactically at 30 mg/kg 2 h before CLP and 8 h after CLP or therapeutically at 10 or 30 mg/kg 2 h and 8 h after CLP. dexamethasone was given orally at 10 mg/kg 1 h before CLP. 10 mice were included in each group.

Evans blue dye (0.1 mL at 40 mg/kg) was injected through each tail vein 30 minutes before euthanasia at 24 hours after CLP. Lung, kidney, liver and heart tissues are collected, and Evans blue was extracted in Formamide. The concentration of the Evans blue dye was calculated from a standard curve and expressed as μg/100 mg lung dry tissue. The data were analyzed by using commercially available software (Prism, version 8.3.0; GraphPad Software Inc., San Diego, CA). Different groups were compared with the one-way variance analysis (ANOVA) followed by the Dunnett test (e.g CLP group compared to treated groups). All data were expressed as a mean±SEM. The limit of the significance was taken as p values less than 0.05 (p<0.05).

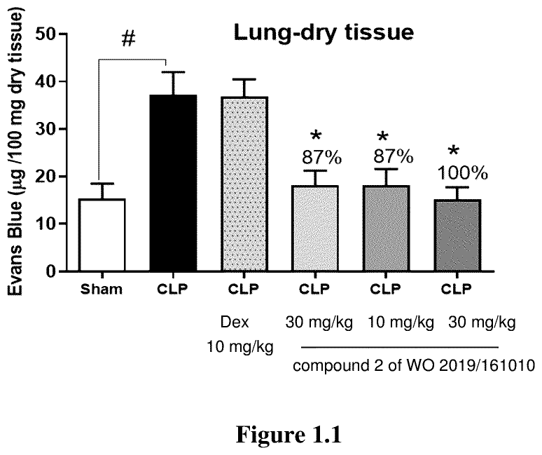

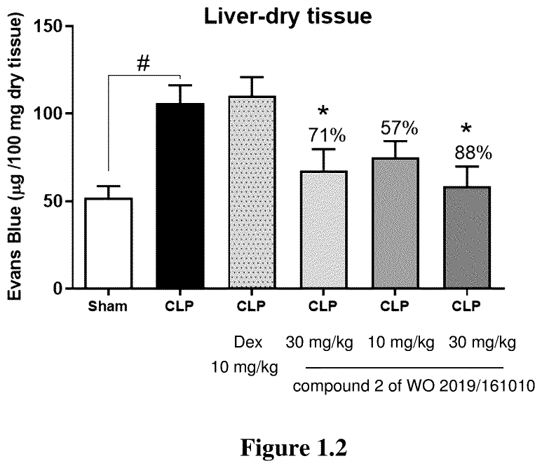

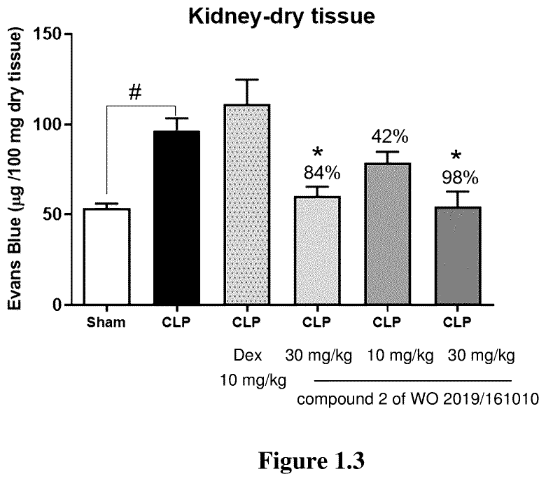

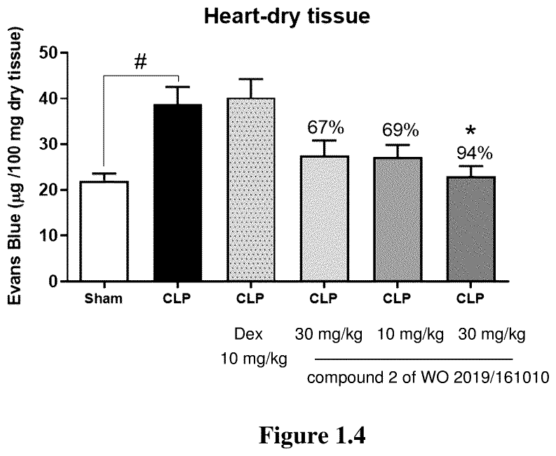

In the lung, Evans blue concentration (37 μg/100 mg dry tissue) in the CLP vehicle-treated group is significantly (p<0.05) higher than Evans blue in the sham group (15 μg/100 mg dry tissue) (FIG. 1.1). Compound 2 of WO 2019/161010 significantly (p<0.05) reduced Evans blue concentration either in prophylactic mode (87% inhibition at 30 mg/kg) or therapeutic mode (87% inhibition at 10 mg/kg and 100% at 30 mg/kg) (FIG. 1.1). Dexamethasone was not able to reduce lung vascular permeability in this model (FIGS. 1.1 to 1.4).

In the liver, Evans blue concentration (106 μg/100 mg dry tissue) in the CLP vehicle-treated group is significantly (p<0.05) higher than Evans blue in the sham group (52 μg/100 mg dry tissue) (FIG. 1.2). Compound 2 of WO 2019/161010 significantly (p<0.05) reduced Evans blue concentration either in prophylactic mode (71% inhibition at 30 mg/kg) or therapeutic mode (88% at 30 mg/kg) (FIG. 1.2). Dexamethasone was not able to reduce lung vascular permeability in this model (FIG. 1.1).

In the kidney, Evans blue concentration (97 μg/100 mg dry tissue) in the CLP vehicle-treated group is significantly (p<0.05) higher than Evans blue in the sham group (53 μg/100 mg dry tissue) (FIG. 1.3). Compound 2 of WO 2019/161010 significantly (p<0.05) reduced Evans blue concentration either in prophylactic mode (84% inhibition at 30 mg/kg) or therapeutic mode (98% inhibition at 30 mg/kg) (FIG. 1.3). Dexamethasone was not able to reduce lung vascular permeability in this model (FIG. 1.3).

In the heart, Evans blue concentration (39 μg/100 mg dry tissue) in the CLP vehicle-treated group is significantly (p<0.05) higher than Evans blue in the sham group (22 μg/100 mg dry tissue) (FIG. 1.4). Compound 2 of WO 2019/161010 significantly (p<0.05) reduced Evans blue concentration in therapeutic mode (94% inhibition at 30 mg/kg) (FIG. 1.4). Dexamethasone (Dex) was not able to reduce lung vascular permeability in this model (FIG. 1.4).

### Example 2: Effect of Compound 17 on Acute Model of CLP

Compound 17 was also tested in the mouse model of CLP under the same experimental conditions used for compound 2 of WO 2019/161010. In this experiment, compound 17 and compound 2 of WO 2019/161010 were given orally in a therapeutic mode, 2 and 8 h after CLP.

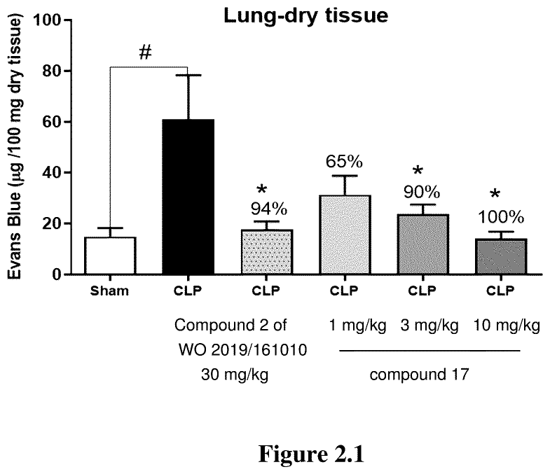

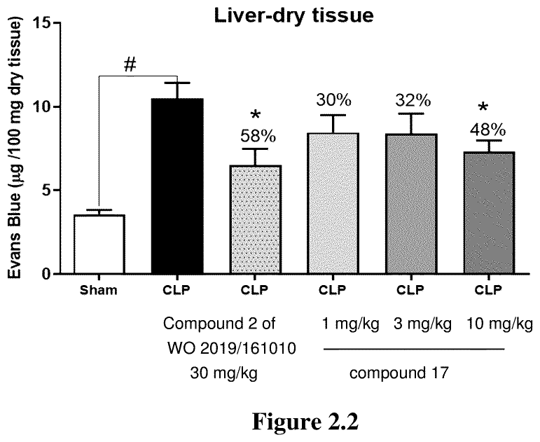

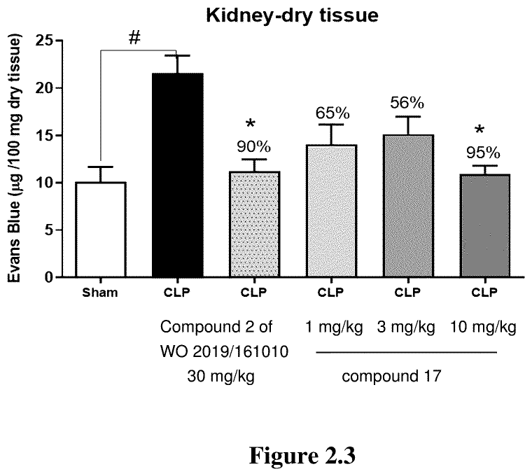

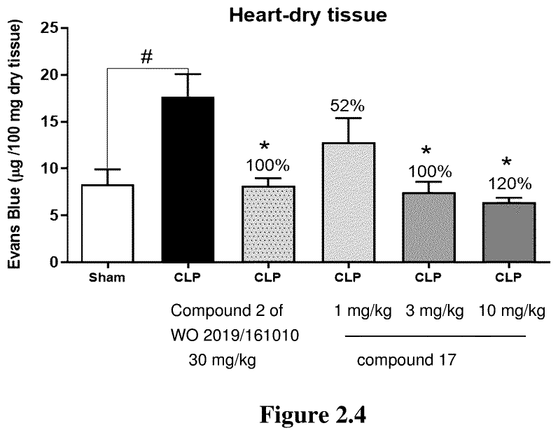

In the lung, Evans blue concentration (61 μg/100 mg dry tissue) in the CLP vehicle-treated group is significantly (p<0.05) higher than Evans blue in the sham group (15 μg/100 mg dry tissue) (FIG. 2.1). Compound 17 significantly (p<0.05) reduced Evans blue concentration dose-dependently (65% inhibition at 1 mg/kg, 90% at 3 mg/kg and 100% at 10 mg/kg) (FIG. 2.1). Compound 2 of WO 2019/161010 significantly reduced Evans blue concentration in lung tissue by 94% at 30 mg/kg (FIG. 2.1).

In the liver, Evans blue concentration (11 μg/100 mg dry tissue) in the CLP vehicle-treated group is significantly (p<0.05) higher than Evans blue in the sham group (4 μg/100 mg dry tissue) (FIG. 2.2). Compound 17 significantly (p<0.05) reduced Evans blue concentration by 48% at 10 mg/kg. Compound 2 of WO 2019/161010 significantly reduced Evans blue concentration by 58% at 30 mg/kg (FIG. 2.2).

In the kidney, Evans blue concentration (22 μg/100 mg dry tissue) in the CLP vehicle-treated group is significantly (p<0.05) higher than Evans blue in the sham group (10 μg/100 mg dry tissue) (FIG. 2.3). Compound 17 significantly (p<0.05) reduced Evans blue concentration by 95% at 10 mg/kg (FIG. 2.3). Compound 2 of WO 2019/161010 significantly reduced Evans blue concentration by 90% at 30 mg/kg (FIG. 2.3).

In the heart, Evans blue concentration (18 μg/100 mg dry tissue) in the CLP vehicle-treated group is significantly (p<0.05) higher than Evans blue in the sham group (8 μg/100 mg dry tissue) (FIG. 2.4). Compound 17 significantly (p<0.05) reduced Evans blue concentration by 100% at 3 mg/kg and 120% at 10 mg/kg (FIG. 2.4). Compound 2 of WO 2019/161010 significantly reduced Evans blue concentration by 100% at 30 mg/kg (FIG. 2.4).

### Example 3: Reduction of LPS-Induced Vascular Leakage in a Mouse Model

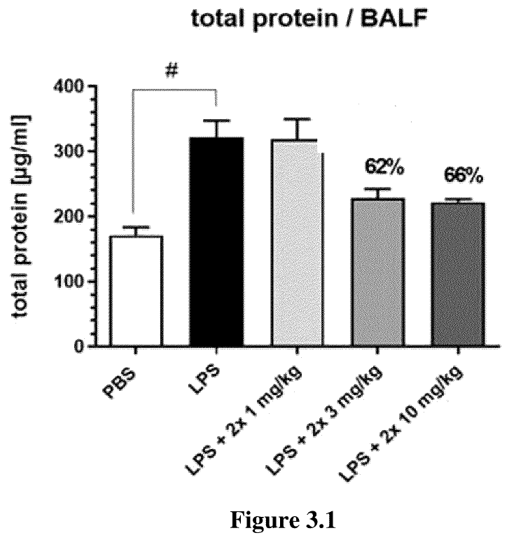

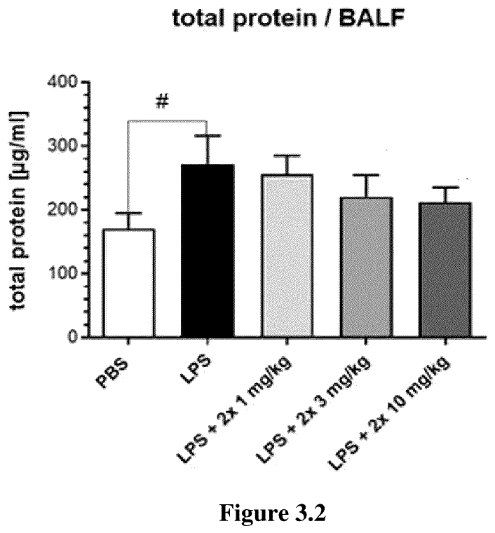

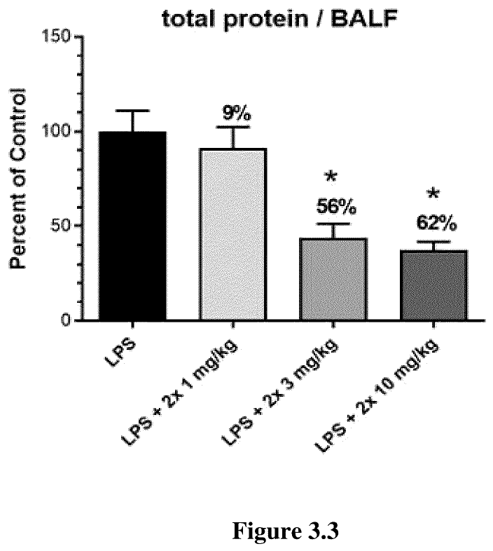

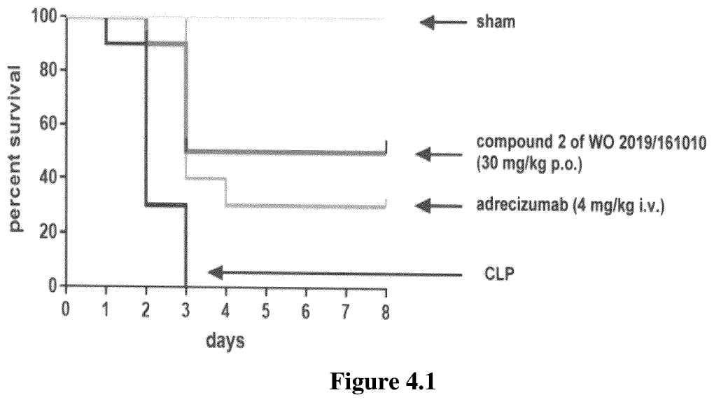

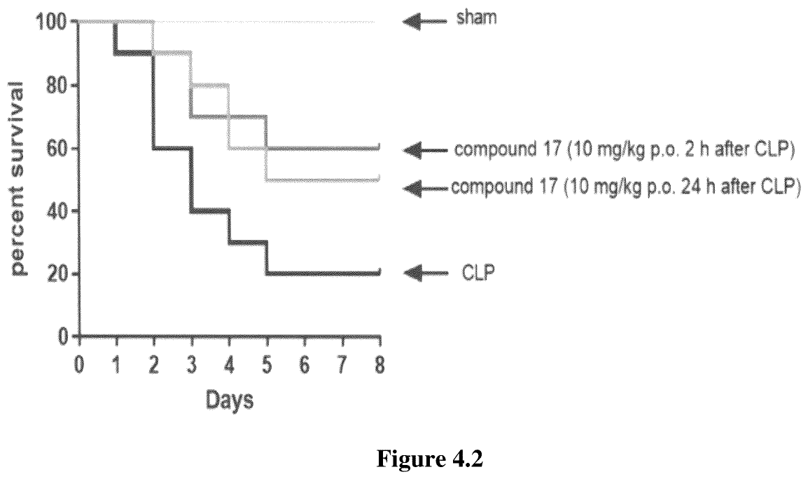

Mice are placed in a chamber and exposed to Lipopolysaccharide (LPS, known as endotoxin and found in outer membrane of Gram-negative bacteria such as Escherichia Coli) aerosol (0.8 mg/ml) for 30 min (or Phosphate-Buffered Saline, PBS as vehicle). The TRPC6 inhibitor compound 17 is given orally 12 h and 2 h before LPS challenge. The mice are euthanized 4 h after the end of the LPS aerosol exposure. Blood is collected for plasma exposure of the compound and the lungs are flushed with 0.8 ml PBS. The broncho-alveolar-lavage is centrifuged at 500 revolutions/min for 10 min and the supernatant is collected for the measurement of total protein according to Lowry measurement by absorbance at 660 nm. The mouse LPS experiment is repeated twice, and the data of each experiment are represented in FIG. 3.1 for the first experiment and FIG. 3.2 for the second experiment. FIG. 3.3 represents the average of the two experiments expressed in percent of the LPS group. Plasma concentration of the compound measured at the end of the experiments is expressed as multiple of in vitro Patch Clamp IC50 (19 nM), IC75 (48 nM), IC90 (104 nM).

LPS aerosol induced lung edema is characterized by a significant accumulation of Broncho-Alveolar-Lavage protein (BALF protein). The origin of these proteins is albumin from the blood due to the vascular hyperpermeability and proteins from the membranes of lung alveolar cells, which are damaged. In the LPS groups, BALF protein (280-310 μg/ml BALF, FIG. 3.1 and FIG. 3.2) is significantly higher than BALF protein in the PBS groups (170-180 μg/ml BALF, FIG. 3.1 and FIG. 3.2). The compound 17 given significantly reduced BALF protein concentration of 56% at 3 mg/kg p.o. and 62% at 10 mg/kg p.o. (FIG. 3.3).

FIG. 3.1 and FIG. 3.2 show the amount and FIG. 3.3 the percent of total protein in BALF (Broncho Alveolar Lavage Fluid) after LPS (LipoPolySaccharide) treatment without or after pretreatment with (+) compound 17 as evaluated along the following tabular scheme

| t [h] | −12 h | -2 h | 0 h | 4 h |
|---|---|---|---|---|
| | Compound 17 | | LPS aerosol | Broncho alveolar |
| | (1, 3, 10 | | (0.8 mg/ml | lavage and blood |
| | mg/kg p.o.) | | 30 min) | sample collection |
| | ↓ | ↓ | ↓ | ↓ |

### Example 4: Chronic Model of CLP: Survival Rate

On day 0, mice were anesthetized with ketamine (80 mg kg⁻¹, i.p.) and xylazine (10 mg kg⁻¹, i.p.). A 1-1.5 cm abdominal midline incision was made, and the caecum was located and tightly ligated at half the distance between distal pole and the base of the caecum with 4-0 silk suture (mild grade). The caecum was punctured through-and-through once with a 21-gauge needle after medium ligation. A small amount of stool was extruded to ensure that the wounds were patent. Then the cecum was replaced in its original position within the abdomen, which was closed with sutures in layers. For negative control animals, a sham surgery was performed: sham-operated animals underwent identical laparotomy but without cecal ligation or puncture. The animals were resuscitated immediately after surgery with 1 mL subcutaneous normal saline and returned to their cages.

Compound 2 of WO 2019/161010 or its vehicle (0.5% natrosol 0.010% Tween 80) was given orally (5 ml/kg) at 30 mg/kg 2 h after CLP and once daily from day 1 to day 6. Adrecizumab, anti-adrenomedullin antibody, was given at 4 mg/kg i.v. only once, 1 h before CLP. 10 mice were included in each group.

No mortality was observed in the sham group over 8 days, while all mice were found dead in the CLP group on day 3. Compound 2 of WO 2019/161010 significantly reduced mortality at day 8 with a survival rate of 50% (5 mice out of 10 were still alive at day 8) (FIG. 4.1). Adrecizumab significantly reduced mortality at day 8 with a survival rate of 30% (3 mice out of 10 were still alive at day 8). Compound 2 of WO 2019/161010 survival rate was significantly superior to adrecizumab survival rate.

FIG. 4.1 compares the effect of compound 2 of WO 2019/161010 on the survival rate in the mouse CLP model with the sham effect and the effect of adrecizumab

In a second experiment, Compound 17 or its vehicle (0.5% natrosol 0.010% Tween 80) was given orally (5 ml/kg). Compound 17 was given at 10 mg/kg in two groups. In the first group, Compound 17 was given at 2 h after CLP and twice daily from day 1 to day 6. In the second group, Compound 17 was given at 24 h after CLP and twice daily from day 2 to day 6. 10 mice were included in each group.

No mortality was observed in the sham group over 8 days, while only 2 mice survived until day 8 in the CLP group. Compound 17 given 2 h after CLP significantly reduced mortality at day 8 with a survival rate of 60% (6 mice out of 10 were still alive at day 8) (FIG. 4.2). The survival rate was still of 50% (5 mice out of 10 mice still alive at day 8), when the compound was given 24 h after CLP.

FIG. 4.2 compares the effect of compound 17 on the survival rate in the mouse CLP model with the sham effect.

1. A method for treating a patient with a systemic inflammatory response to a bacterial or fungal infection, the method comprising administering to the patient a pharmaceutically effective amount of a compound of formula (I), 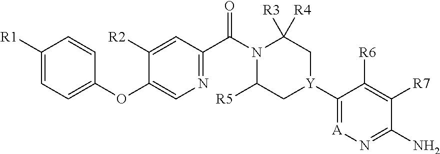
wherein
Y is CH or N,
A is CH or N,
R1 is selected from the group consisting of methyl, ethyl and propyl in which the hydrogen atoms may be partially or fully replaced by fluorine,
or R1 is selected from the group consisting of halogen, C₃₋₆-cycloalkyl, OC₃₋₆-cycloalkyl, and OC₁₋₆-alkyl, wherein the alkyl groups may optionally be substituted with 1 to 3 halogen, and
C₃₋₆-cycloalkyl optionally substituted with 1 to 3 groups independently selected from the group consisting of halogen and C₁₋₆-alkyl optionally substituted with 1 to 3 halogen,
R2 is selected from the group consisting of H, C₁₋₆-alkyl such as —CH₃ and —CH₂CH₃, OCF₃, C₃₋₆-cycloalkyl, OC₁₋₆-alkyl, and OC₃₋₆-cycloalkyl,
R3 is selected from the group consisting of H, C₁₋₆-alkyl, C₃₋₆-cycloalkyl, and OC₃₋₆-cycloalkyl; wherein each of the C₁₋₆-alkyl, C₃₋₆-cycloalkyl, OC₃₋₆-cycloalkyl of the R³ group may be optionally substituted with one to three groups each independently selected from the group consisting of halogen, OH, OC₁₋₆alkyl, SC₁₋₆-alkyl, and N(C₁₋₆-alky)₂; and wherein one to three carbon atoms of the C₁₋₆-alkyl of the R3 group may optionally be replaced with one or two moieties selected from the group consisting of NH, N(C₁₋₆-alkyl), O, and S,
R4 and R5 are each independently selected from the group consisting of H and C₁₋₆-alkyl,
R3 and R4 can together with the atom to which they are attached join to form a 3 to 9-membered carbocyclyl ring which optionally may contain one to three heteroatoms selected from the group consisting of N, O, and S, or
R3 and R5 can together with the atoms to which they are attached join to form a 3 to 9-membered bicyclic ring which optionally may contain one to three heteroatoms selected from the group consisting of N, O, and S,
R6 is selected from the group consisting of H, C₁₋₆-alkyl, CN, CF₃, OCF₃, C₃₋₆-cycloalkyl, OC₁₋₆-alkyl, and OC₃₋₆-cycloalkyl,
R7 is selected from the group consisting of H and OC₁₋₆-alkyl,
or a pharmaceutically acceptable salt thereof together with one or more pharmaceutically acceptable carrier.

2. The method of claim 1, wherein
R1 of formula (I) is selected from the group consisting of CF₃, OCF₃, halogen, OC₃₋₆-cycloalkyl, and OC₁₋₆-alkyl optionally substituted with one to three halogen, and C₃₋₆-cycloalkyl optionally substituted with 1 to 3 halogen groups,
R2 of formula (I) is OC₁₋₆-alkyl,
R3 of formula (I) is selected from the group consisting of H and C₁₋₆-alkyl optionally substituted with OH or OC₁₋₆-alkyl,
R4 of formula (I) is H,
R5 of formula (I) is H,
R3 and R4 of formula (I) can together with the atom to which they are attached join to form a 3 to 9-membered carbocyclyl ring which optionally may contain one to three heteroatoms selected from the group consisting of N and O, or
R3 and R5 of formula (I) can together with the atoms to which they are attached join to form a 3 to 9-membered bicyclic which optionally may contain one to three heteroatoms selected from the group consisting of N and O,
R6 of formula (I) is selected from the group consisting of H, C₁₋₆-alkyl, OC₁₋₆-alkyl, and OC₃₋₆-cycloalkyl,
R7 of formula (I) is selected from the group consisting of H and OC₁₋₆-alkyl.

3. The method of claim 1, wherein the comprising a compound of formula (I) has having the structure, 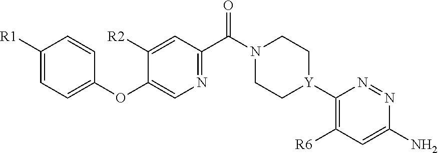
wherein
Y is CH or N,
R1 is selected from the group consisting of:
CF₃, halogen, OC₃₋₆-cycloalkyl, and OC₁₋₆-alkyl optionally substituted with one to three halogen, and
unsubstituted cyclohexyl or cyclohexyl substituted with a group selected from the group consisting of fluorine (F), unsubstituted —CH₃, —CH₃ substituted with 1-3 fluoro atoms, unsubstituted —CH₂CH₃, —CH₂CH₃ substituted with 1-5 fluoro atoms, unsubstituted propyl and propyl substituted with 1-7 fluoro atoms,
R2 is selected from the group consisting of H, —CH₃, —CH₂CH₃, cyclopropyl, cyclobutyl, cyclopentyl, cyclohexyl, and —OC₁₋₆-alkyl such as —O—CH₃, —O—CH₂CH₃, —O—CF₃, hydroxymethyl, hydroxyethyl and hydroxypropyl, and
R6 is selected from the group consisting of hydrogen, unsubstituted methyl, unsubstituted ethyl, unsubstituted propyl, methyl substituted with 1-3 fluoro atoms, ethyl substituted with 1-5 fluoro atoms and propyl substituted with 1-7 fluoro atoms,
R7 is selected from the group consisting of H and —OC₁₋₆-alkyl,
or a pharmaceutically acceptable salt thereof.

4. The method of claim 1, wherein the compound of formula (I) has the structure, 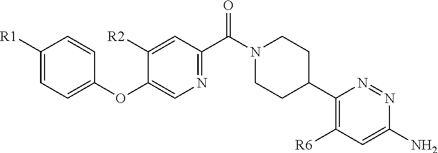
wherein
R1 is unsubstituted methyl, ethyl or propyl or methyl, ethyl or propyl substituted with 1-7 fluorine atoms, or fluorine,
R2 is selected from OC₁₋₆-alkyl such as methoxy, ethoxy and propoxy,
R6 is selected from the group consisting of H, unsubstituted methyl, unsubstituted ethyl and unsubstituted propyl, methyl substituted with 1-3 fluoro atoms, ethyl substituted with 1-5 fluoro atoms, and propyl substituted with 1-7 fluoro atoms; methoxy, ethoxy, propoxy and cyclylpropyloxy,
or a pharmaceutically acceptable salt thereof.

5. The method of claim 1, wherein the compound of formula (I) is selected from the group consisting of: 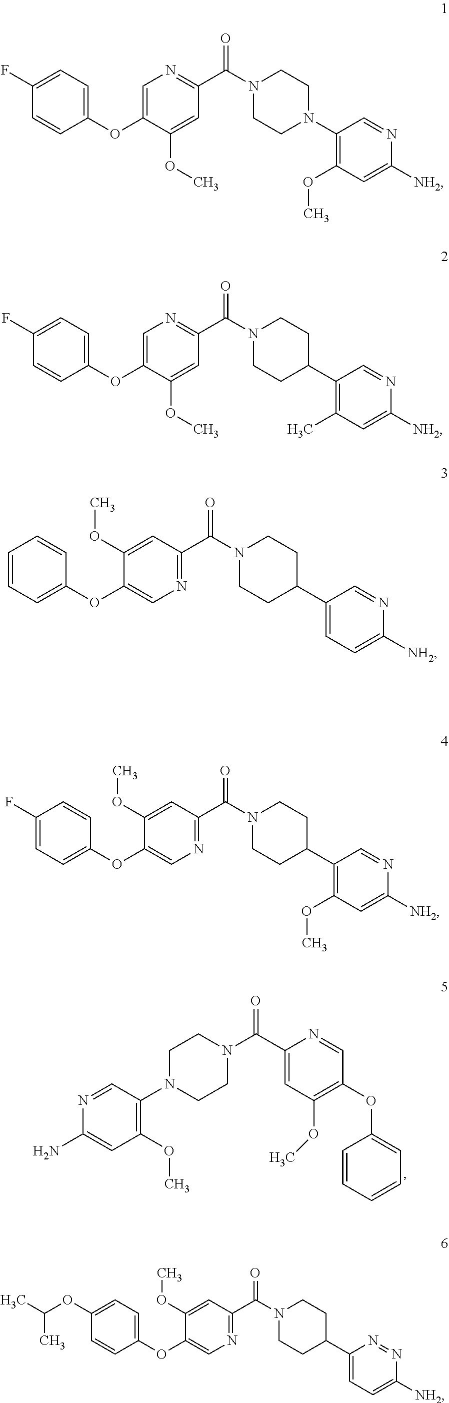 [SMILES: COc1cc(C(=O)N2CCN(c3cnc(N)cc3OC)CC2)ncc1Oc1ccc(F)cc1 ; COc1cc(C(=O)N2CCC(c3cnc(N)cc3C)CC2)ncc1Oc1ccc(F)cc1 ; COc1cc(C(=O)N2CCC(c3ccc(N)nc3)CC2)ncc1Oc1ccccc1 ; COc1cc(C(=O)N2CCC(c3cnc(N)cc3OC)CC2)ncc1Oc1ccc(F)cc1 ; COc1cc(C(=O)N2CCN(c3cnc(N)cc3OC)CC2)ncc1Oc1ccccc1 ; COc1cc(C(=O)N2CCC(c3ccc(N)nn3)CC2)ncc1Oc1ccc(OC(C)C)cc1] 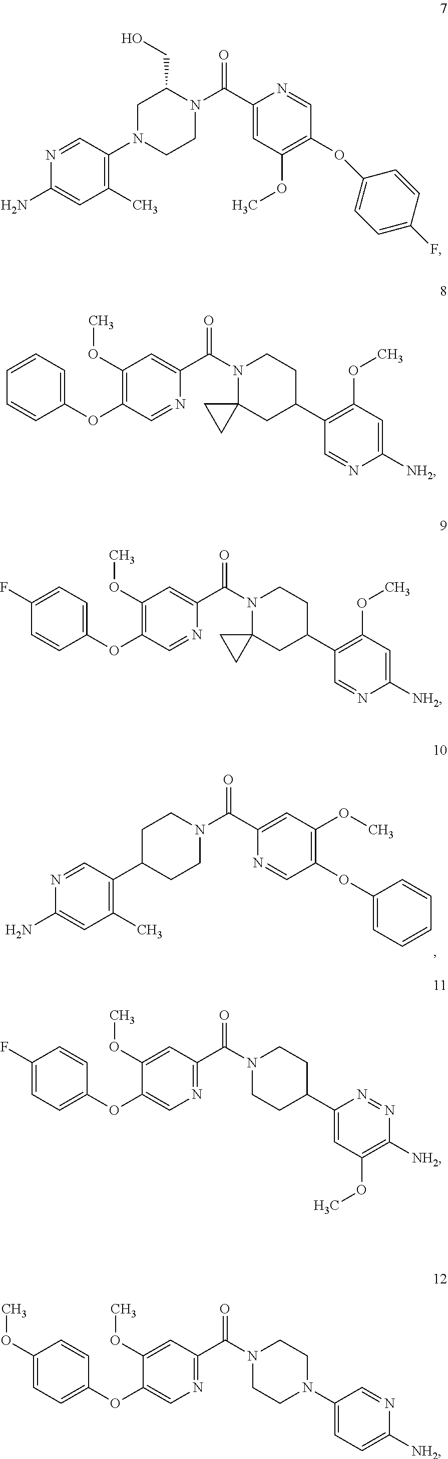 [SMILES: COc1cc(C(=O)N2CCN(c3cnc(N)cc3C)C[C@@H]2CO)ncc1Oc1ccc(F)cc1 ; COc1cc(C(=O)N2CCC(c3cnc(N)cc3OC)CC23CC3)ncc1Oc1ccccc1 ; COc1cc(C(=O)N2CCC(c3cnc(N)cc3OC)CC23CC3)ncc1Oc1ccc(F)cc1 ; COc1cc(C(=O)N2CCC(c3cnc(N)cc3C)CC2)ncc1Oc1ccccc1 ; COc1cc(C(=O)N2CCC(c3cc(OC)c(N)nn3)CC2)ncc1Oc1ccc(F)cc1 ; COc1ccc(Oc2cnc(C(=O)N3CCN(c4ccc(N)nc4)CC3)cc2OC)cc1] 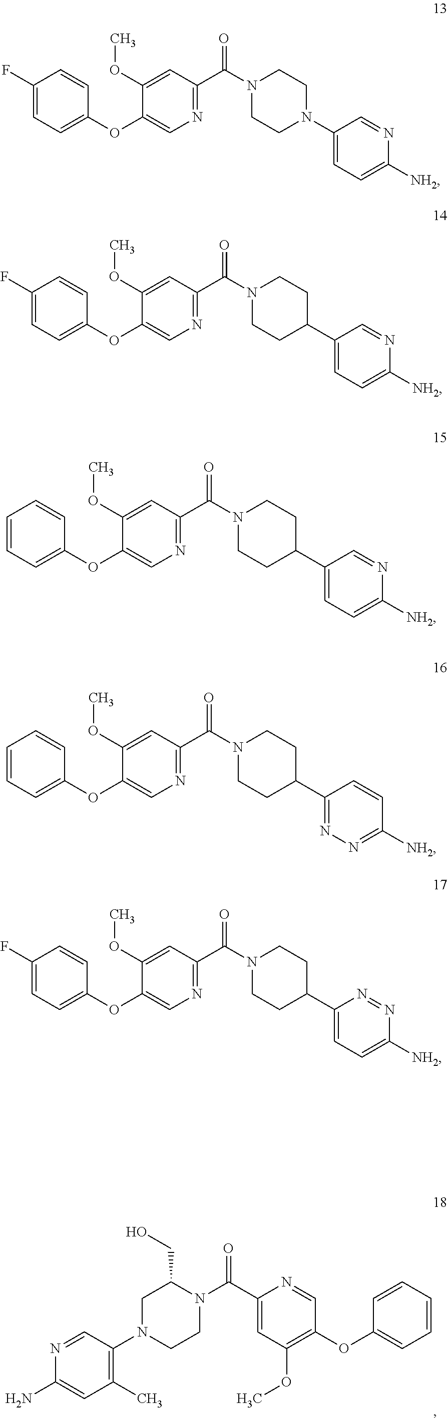 [SMILES: COc1cc(C(=O)N2CCN(c3ccc(N)nc3)CC2)ncc1Oc1ccc(F)cc1 ; COc1cc(C(=O)N2CCC(c3ccc(N)nc3)CC2)ncc1Oc1ccc(F)cc1 ; COc1cc(C(=O)N2CCC(c3ccc(N)nc3)CC2)ncc1Oc1ccccc1 ; COc1cc(C(=O)N2CCC(c3ccc(N)nn3)CC2)ncc1Oc1ccccc1 ; COc1cc(C(=O)N2CCC(c3ccc(N)nn3)CC2)ncc1Oc1ccc(F)cc1 ; COc1cc(C(=O)N2CCN(c3cnc(N)cc3C)C[C@@H]2CO)ncc1Oc1ccccc1] 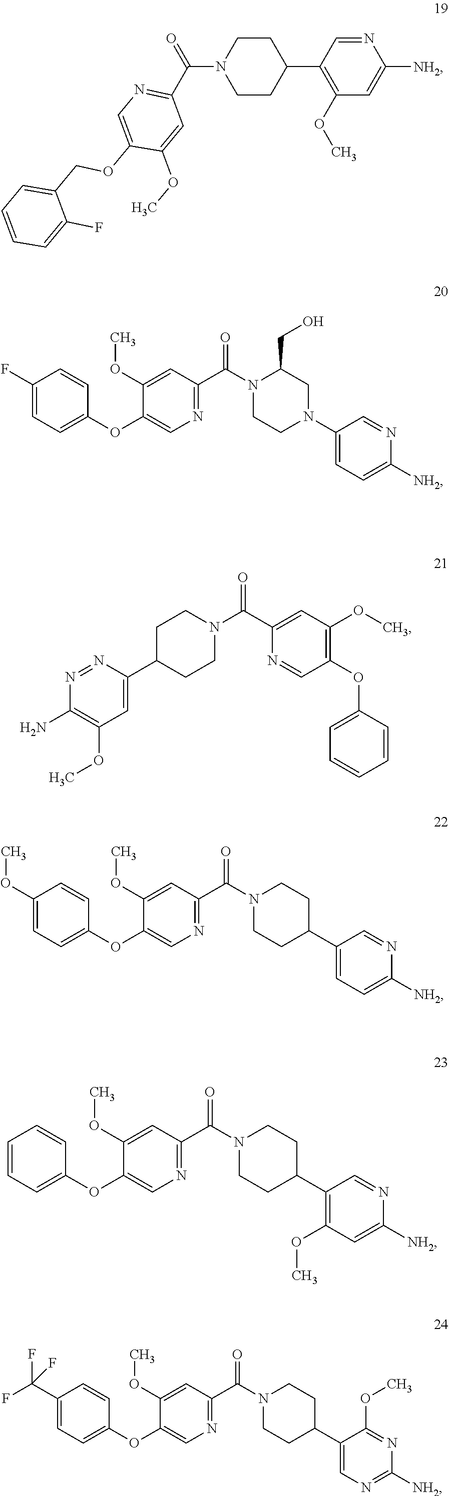 [SMILES: COc1cc(C(=O)N2CCC(c3cnc(N)cc3OC)CC2)ncc1OCc1ccccc1F ; COc1cc(C(=O)N2CCN(c3ccc(N)nc3)C[C@@H]2CO)ncc1Oc1ccc(F)cc1 ; COc1cc(C(=O)N2CCC(c3cc(OC)c(N)nn3)CC2)ncc1Oc1ccccc1 ; COc1ccc(Oc2cnc(C(=O)N3CCC(c4ccc(N)nc4)CC3)cc2OC)cc1 ; COc1cc(C(=O)N2CCC(c3cnc(N)cc3OC)CC2)ncc1Oc1ccccc1 ; COc1cc(C(=O)N2CCC(c3cnc(N)nc3OC)CC2)ncc1Oc1ccc(C(F)(F)F)cc1] 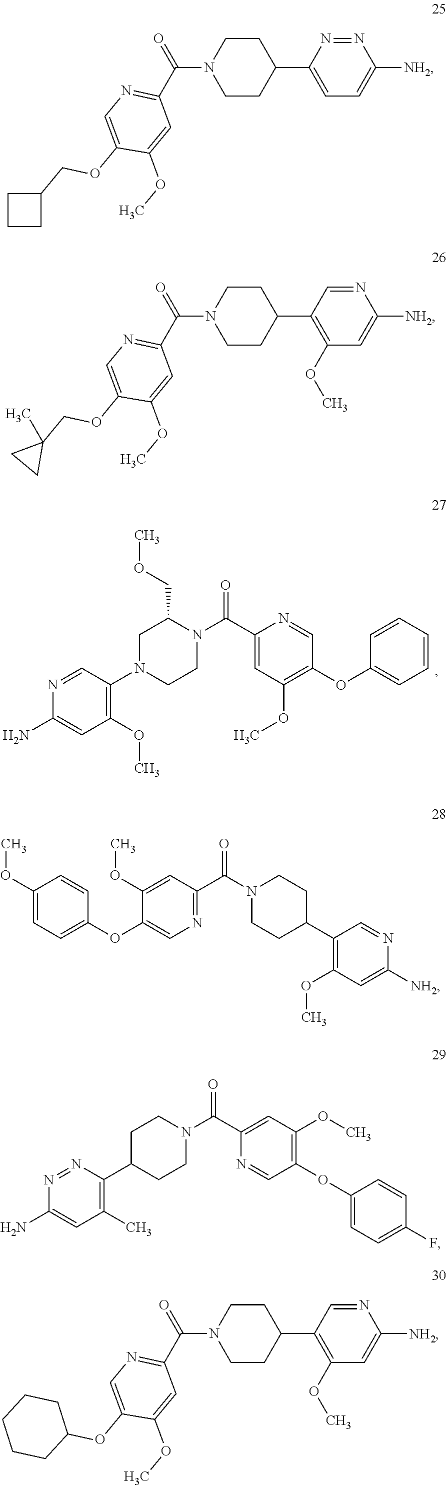 [SMILES: COc1cc(C(=O)N2CCC(c3ccc(N)nn3)CC2)ncc1OCC1CCC1 ; COc1cc(C(=O)N2CCC(c3cnc(N)cc3OC)CC2)ncc1OCC1(C)CC1 ; COC[C@H]1CN(c2cnc(N)cc2OC)CCN1C(=O)c1cc(OC)c(Oc2ccccc2)cn1 ; COc1ccc(Oc2cnc(C(=O)N3CCC(c4cnc(N)cc4OC)CC3)cc2OC)cc1 ; COc1cc(C(=O)N2CCC(c3nnc(N)cc3C)CC2)ncc1Oc1ccc(F)cc1 ; COc1cc(C(=O)N2CCC(c3cnc(N)cc3OC)CC2)ncc1OC1CCCCC1] 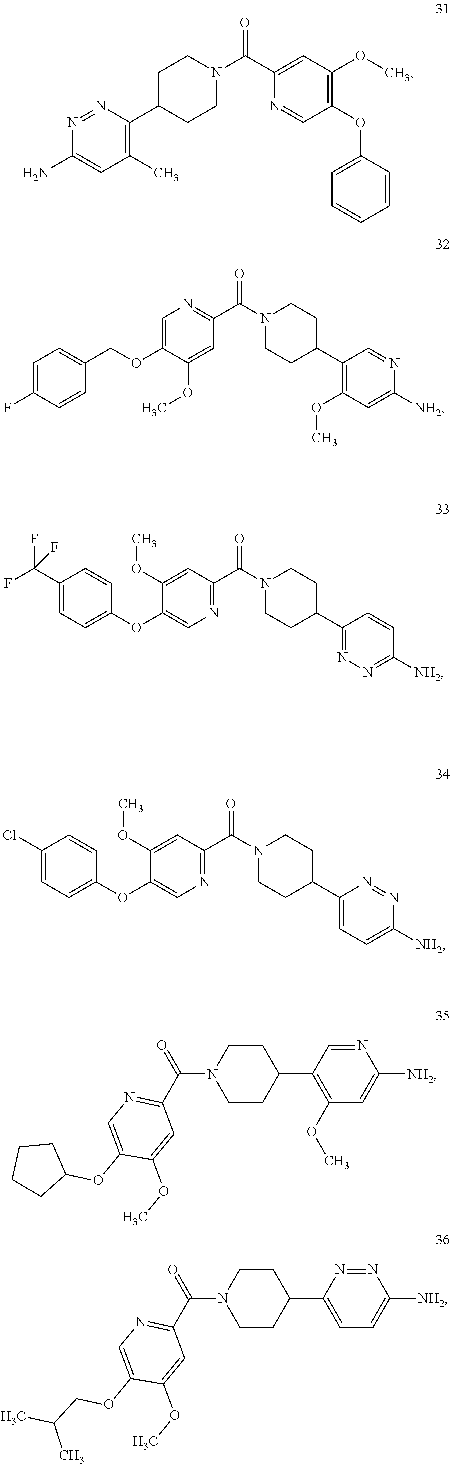 [SMILES: COc1cc(C(=O)N2CCC(c3nnc(N)cc3C)CC2)ncc1Oc1ccccc1 ; COc1cc(C(=O)N2CCC(c3cnc(N)cc3OC)CC2)ncc1OCc1ccc(F)cc1 ; COc1cc(C(=O)N2CCC(c3ccc(N)nn3)CC2)ncc1Oc1ccc(C(F)(F)F)cc1 ; COc1cc(C(=O)N2CCC(c3ccc(N)nn3)CC2)ncc1Oc1ccc(Cl)cc1 ; COc1cc(C(=O)N2CCC(c3cnc(N)cc3OC)CC2)ncc1OC1CCCC1 ; COc1cc(C(=O)N2CCC(c3ccc(N)nn3)CC2)ncc1OCC(C)C] 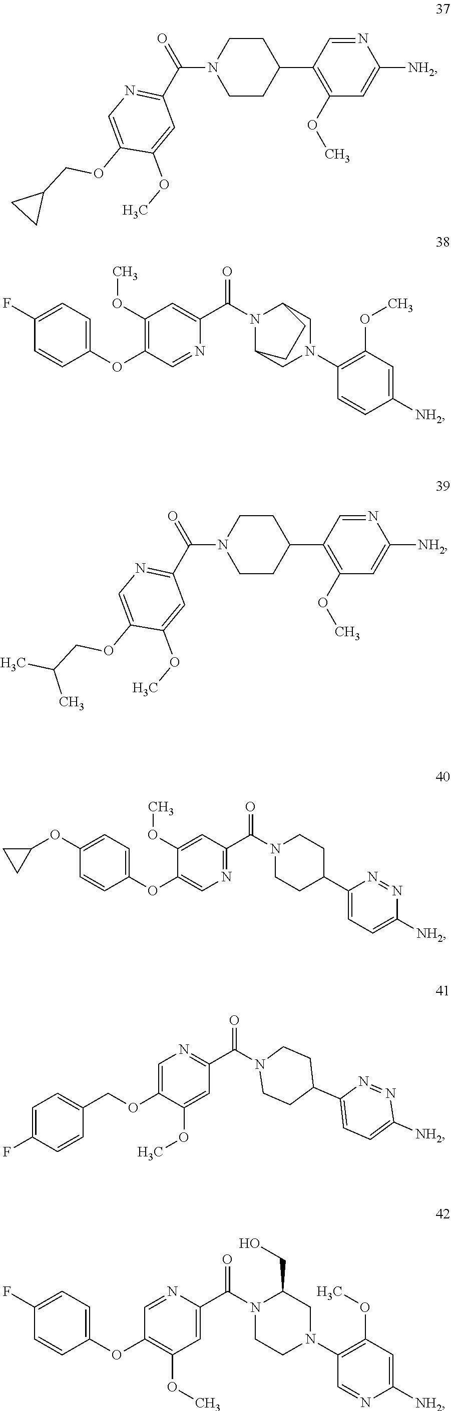 [SMILES: COc1cc(C(=O)N2CCC(c3cnc(N)cc3OC)CC2)ncc1OCC1CC1 ; COc1cc(C(=O)N2C3CCC2CN(c2ccc(N)cc2OC)C3)ncc1Oc1ccc(F)cc1 ; COc1cc(C(=O)N2CCC(c3cnc(N)cc3OC)CC2)ncc1OCC(C)C ; COc1cc(C(=O)N2CCC(c3ccc(N)nn3)CC2)ncc1Oc1ccc(OC2CC2)cc1 ; COc1cc(C(=O)N2CCC(c3ccc(N)nn3)CC2)ncc1OCc1ccc(F)cc1 ; COc1cc(C(=O)N2CCN(c3cnc(N)cc3OC)C[C@@H]2CO)ncc1Oc1ccc(F)cc1] 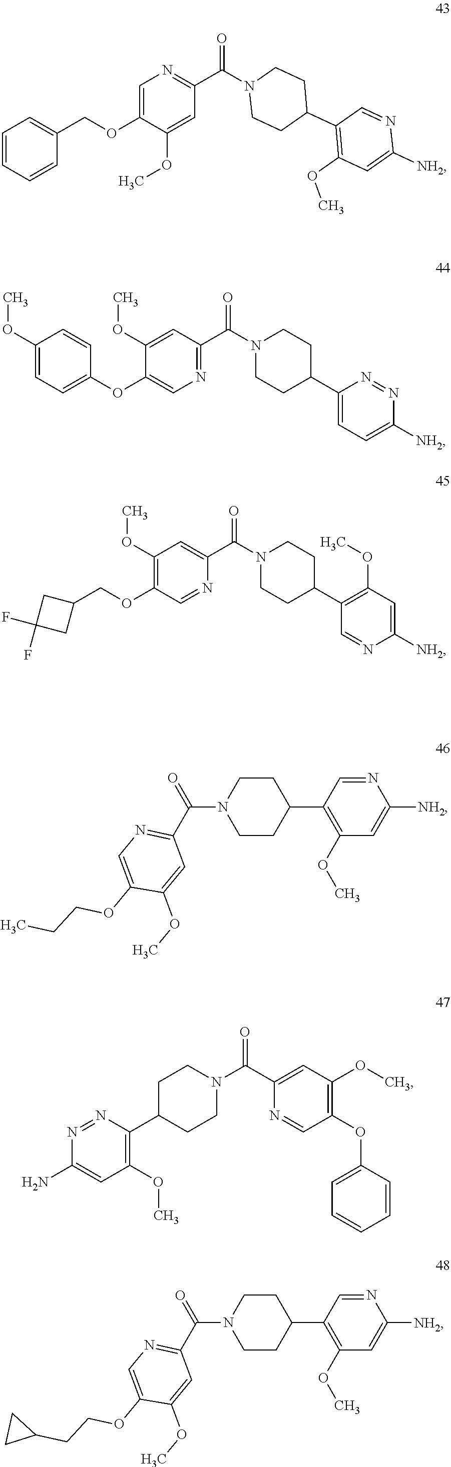 [SMILES: COc1cc(C(=O)N2CCC(c3cnc(N)cc3OC)CC2)ncc1OCc1ccccc1 ; COc1ccc(Oc2cnc(C(=O)N3CCC(c4ccc(N)nn4)CC3)cc2OC)cc1 ; COc1cc(C(=O)N2CCC(c3cnc(N)cc3OC)CC2)ncc1OCC1CC(F)(F)C1 ; CCCOc1cnc(C(=O)N2CCC(c3cnc(N)cc3OC)CC2)cc1OC ; COc1cc(C(=O)N2CCC(c3nnc(N)cc3OC)CC2)ncc1Oc1ccccc1 ; COc1cc(C(=O)N2CCC(c3cnc(N)cc3OC)CC2)ncc1OCCC1CC1] 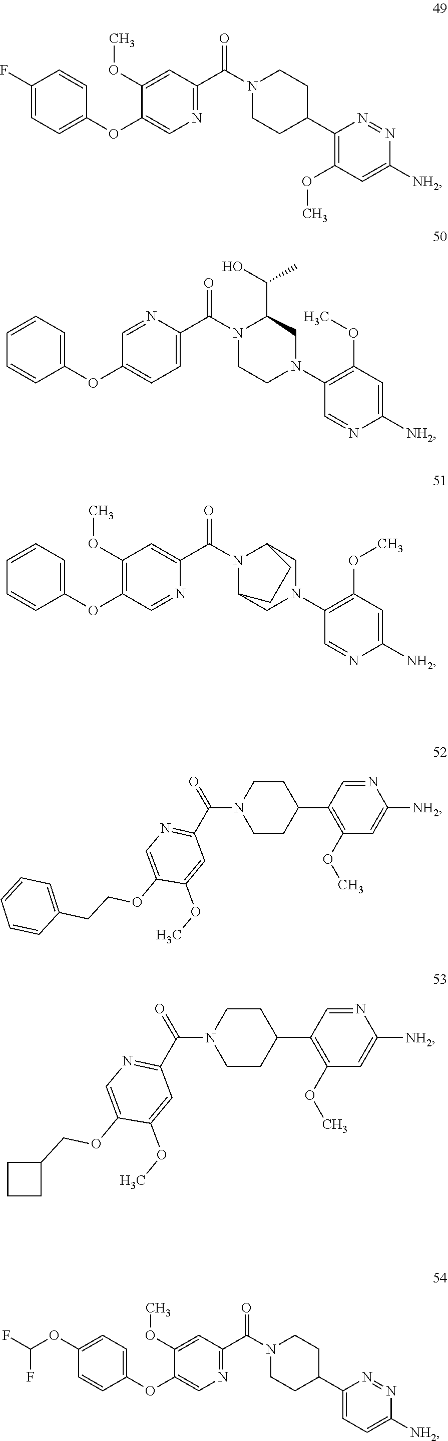 [SMILES: COc1cc(C(=O)N2CCC(c3nnc(N)cc3OC)CC2)ncc1Oc1ccc(F)cc1 ; COc1cc(N)ncc1N1CCN(C(=O)c2ccc(Oc3ccccc3)cn2)[C@@H]([C@@H](C)O)C1 ; COc1cc(C(=O)N2C3CCC2CN(c2cnc(N)cc2OC)C3)ncc1Oc1ccccc1 ; COc1cc(C(=O)N2CCC(c3cnc(N)cc3OC)CC2)ncc1OCCc1ccccc1 ; COc1cc(C(=O)N2CCC(c3cnc(N)cc3OC)CC2)ncc1OCC1CCC1 ; COc1cc(C(=O)N2CCC(c3ccc(N)nn3)CC2)ncc1Oc1ccc(OC(F)F)cc1] 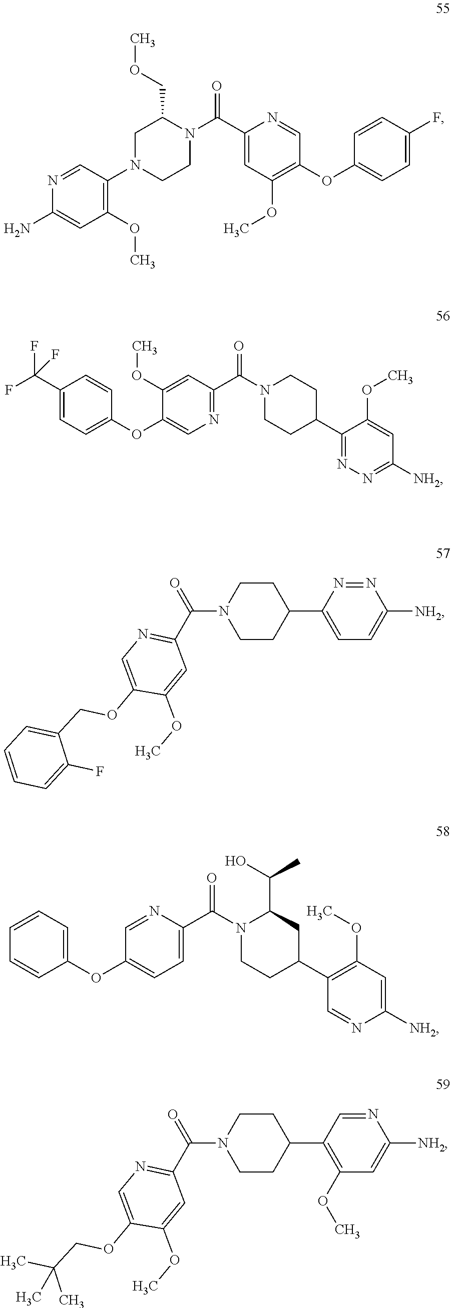 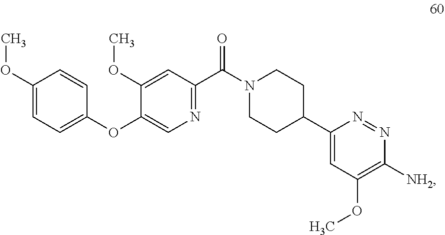 [SMILES: COc1ccc(Oc2cnc(C(=O)N3CCC(c4cc(OC)c(N)nn4)CC3)cc2OC)cc1] 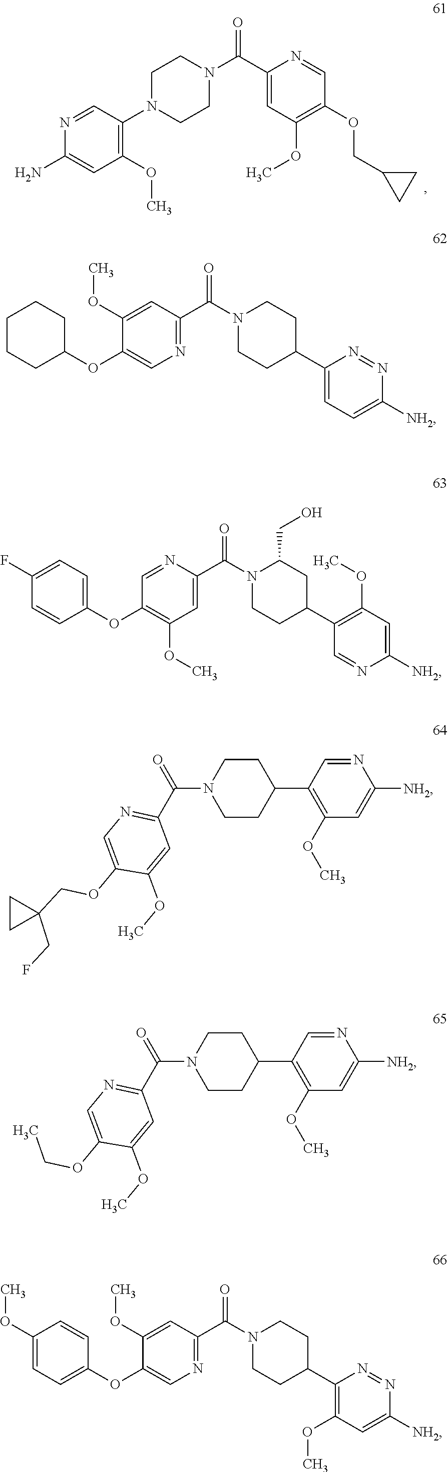 [SMILES: COc1cc(C(=O)N2CCN(c3cnc(N)cc3OC)CC2)ncc1OCC1CC1 ; COc1cc(C(=O)N2CCC(c3ccc(N)nn3)CC2)ncc1OC1CCCCC1 ; COc1cc(C(=O)N2CCC(c3cnc(N)cc3OC)C[C@H]2CO)ncc1Oc1ccc(F)cc1 ; COc1cc(C(=O)N2CCC(c3cnc(N)cc3OC)CC2)ncc1OCC1(CF)CC1 ; CCOc1cnc(C(=O)N2CCC(c3cnc(N)cc3OC)CC2)cc1OC ; COc1ccc(Oc2cnc(C(=O)N3CCC(c4nnc(N)cc4OC)CC3)cc2OC)cc1] 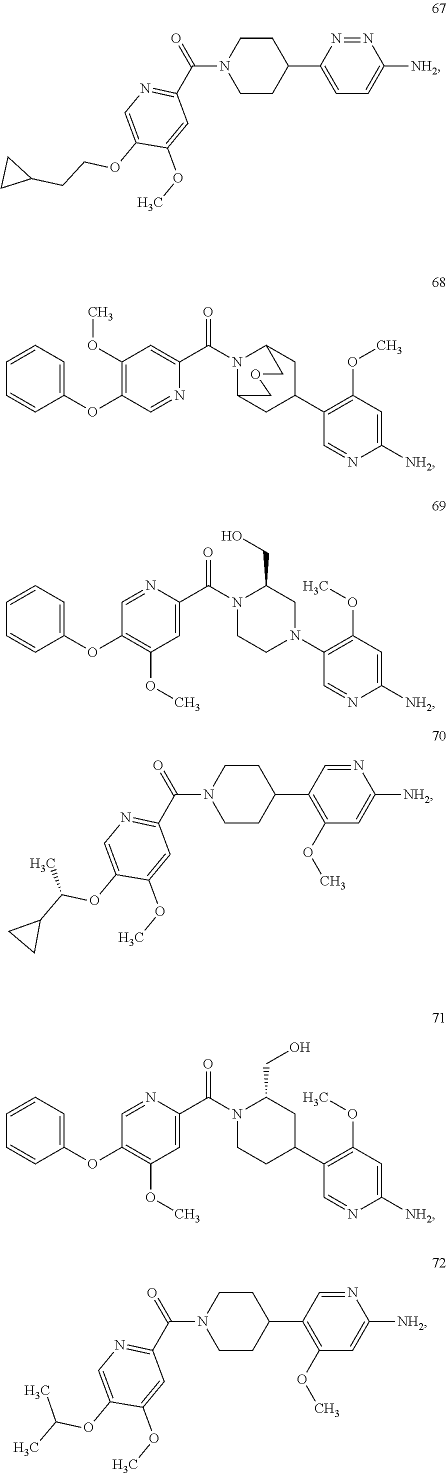 [SMILES: COc1cc(C(=O)N2CCC(c3ccc(N)nn3)CC2)ncc1OCCC1CC1 ; COc1cc(C(=O)N2C3COCC2CC(c2cnc(N)cc2OC)C3)ncc1Oc1ccccc1 ; COc1cc(C(=O)N2CCN(c3cnc(N)cc3OC)C[C@@H]2CO)ncc1Oc1ccccc1 ; COc1cc(C(=O)N2CCC(c3cnc(N)cc3OC)CC2)ncc1O[C@@H](C)C1CC1 ; COc1cc(C(=O)N2CCC(c3cnc(N)cc3OC)C[C@H]2CO)ncc1Oc1ccccc1 ; COc1cc(C(=O)N2CCC(c3cnc(N)cc3OC)CC2)ncc1OC(C)C] 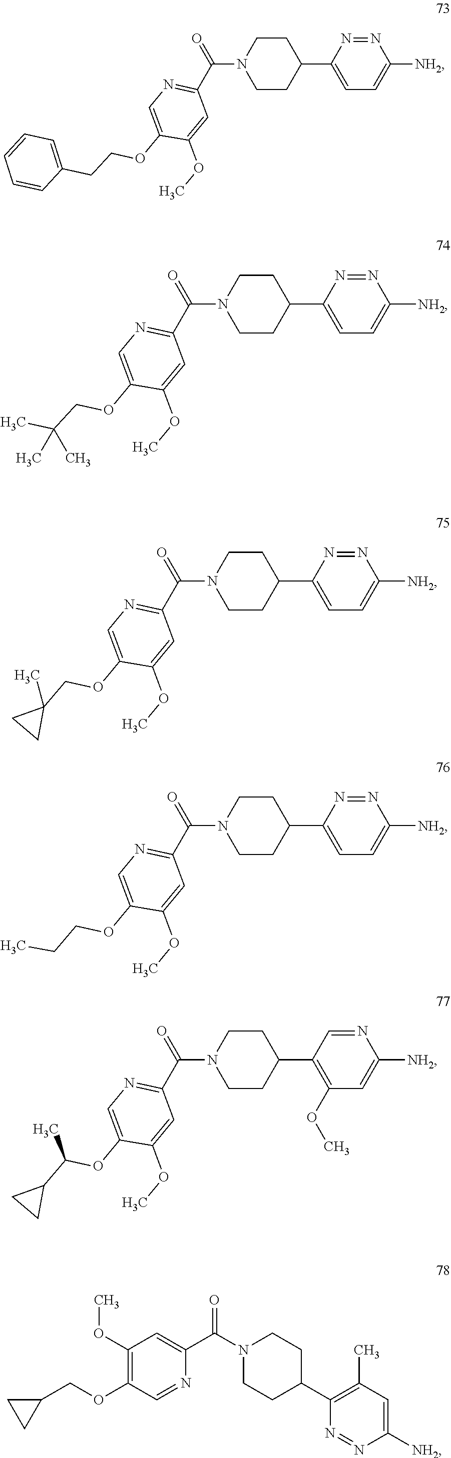 [SMILES: COc1cc(C(=O)N2CCC(c3ccc(N)nn3)CC2)ncc1OCCc1ccccc1 ; COc1cc(C(=O)N2CCC(c3ccc(N)nn3)CC2)ncc1OCC(C)(C)C ; COc1cc(C(=O)N2CCC(c3ccc(N)nn3)CC2)ncc1OCC1(C)CC1 ; CCCOc1cnc(C(=O)N2CCC(c3ccc(N)nn3)CC2)cc1OC ; COc1cc(C(=O)N2CCC(c3cnc(N)cc3OC)CC2)ncc1O[C@H](C)C1CC1 ; COc1cc(C(=O)N2CCC(c3nnc(N)cc3C)CC2)ncc1OCC1CC1] 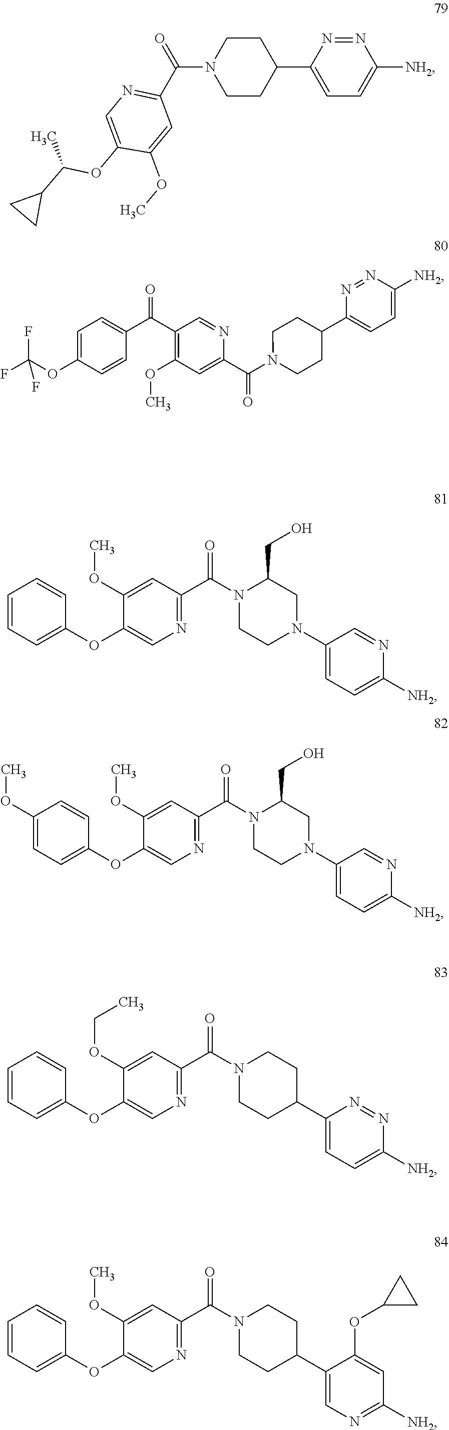 [SMILES: COc1cc(C(=O)N2CCC(c3ccc(N)nn3)CC2)ncc1O[C@@H](C)C1CC1 ; COc1cc(C(=O)N2CCC(c3ccc(N)nn3)CC2)ncc1C(=O)c1ccc(OC(F)(F)F)cc1 ; COc1cc(C(=O)N2CCN(c3ccc(N)nc3)C[C@@H]2CO)ncc1Oc1ccccc1 ; COc1ccc(Oc2cnc(C(=O)N3CCN(c4ccc(N)nc4)C[C@@H]3CO)cc2OC)cc1 ; CCOc1cc(C(=O)N2CCC(c3ccc(N)nn3)CC2)ncc1Oc1ccccc1 ; COc1cc(C(=O)N2CCC(c3cnc(N)cc3OC3CC3)CC2)ncc1Oc1ccccc1] 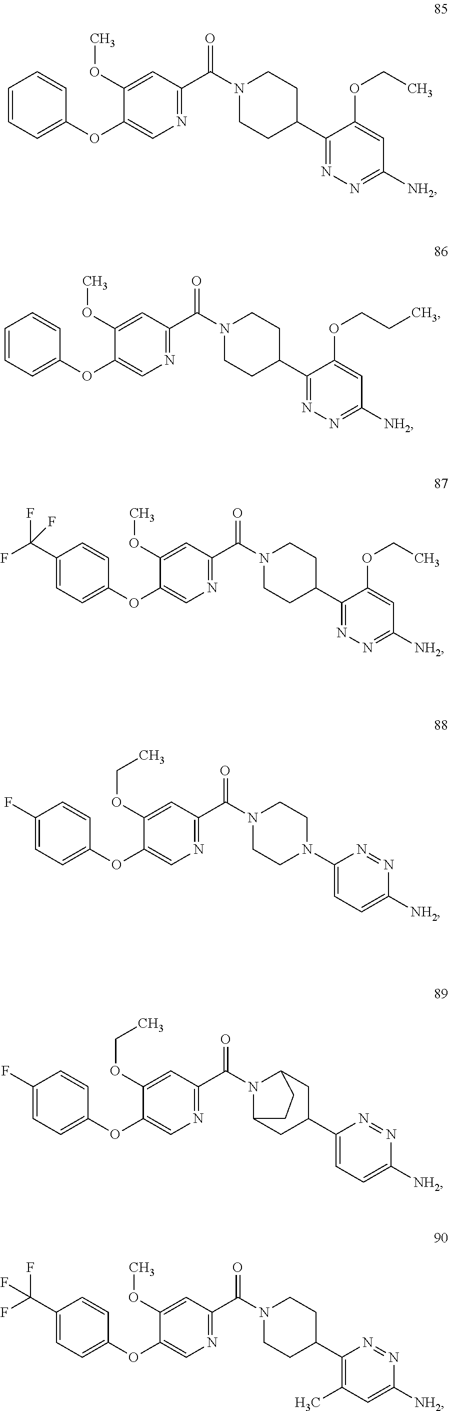 [SMILES: CCOc1cc(N)nnc1C1CCN(C(=O)c2cc(OC)c(Oc3ccccc3)cn2)CC1 ; CCCOc1cc(N)nnc1C1CCN(C(=O)c2cc(OC)c(Oc3ccccc3)cn2)CC1 ; CCOc1cc(N)nnc1C1CCN(C(=O)c2cc(OC)c(Oc3ccc(C(F)(F)F)cc3)cn2)CC1 ; CCOc1cc(C(=O)N2CCN(c3ccc(N)nn3)CC2)ncc1Oc1ccc(F)cc1 ; CCOc1cc(C(=O)N2C3CCC2CC(c2ccc(N)nn2)C3)ncc1Oc1ccc(F)cc1 ; COc1cc(C(=O)N2CCC(c3nnc(N)cc3C)CC2)ncc1Oc1ccc(C(F)(F)F)cc1] 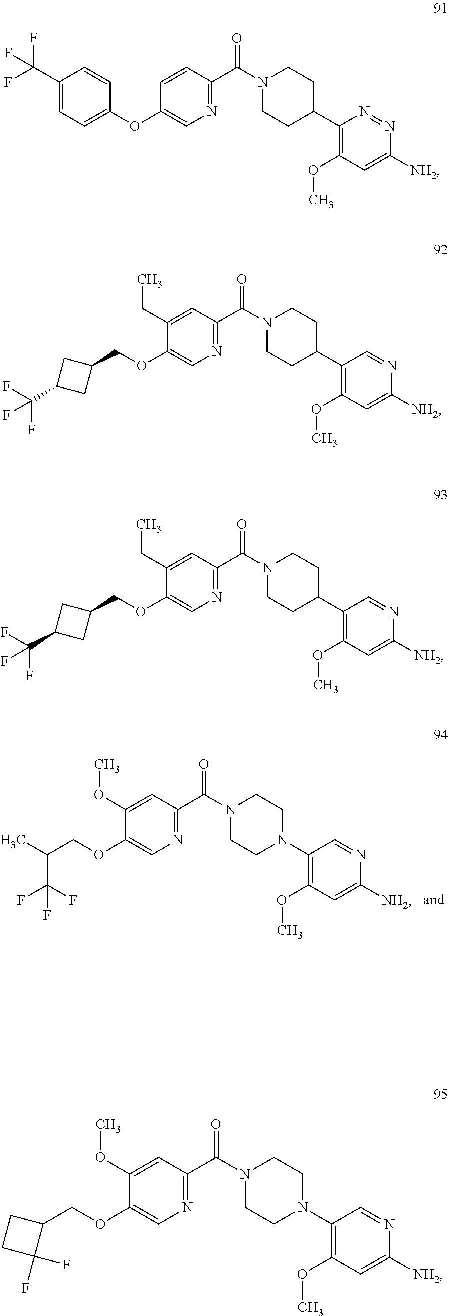 [SMILES: COc1cc(N)nnc1C1CCN(C(=O)c2ccc(Oc3ccc(C(F)(F)F)cc3)cn2)CC1 ; CCc1cc(C(=O)N2CCC(c3cnc(N)cc3OC)CC2)ncc1OC[C@H]1C[C@H](C(F)(F)F)C1 ; CCc1cc(C(=O)N2CCC(c3cnc(N)cc3OC)CC2)ncc1OC[C@H]1C[C@@H](C(F)(F)F)C1 ; COc1cc(C(=O)N2CCN(c3cnc(N)cc3OC)CC2)ncc1OCC(C)C(F)(F)F ; COc1cc(C(=O)N2CCN(c3cnc(N)cc3OC)CC2)ncc1OCC1CCC1(F)F]
or a pharmaceutically acceptable salts thereof.

6. The method of claim 1, wherein the patient's response is characterized by interstitial fluid accumulation and/or hypotension.

7. A method for treating a patient with a systemic inflammatory response to a bacterial or fungal infection, the method comprising administering to the patient a pharmaceutical composition comprising a compound of formula (I). 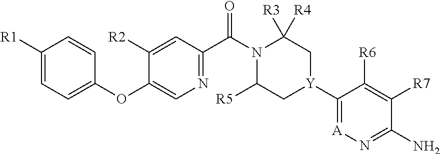
wherein
Y is CH or N,
A is CH or N,
R1 is selected from the group consisting of
methyl, ethyl and propyl in which the hydrogen atoms may be partially or fully replaced by fluorine,
or R1 is selected from the group consisting of halogen, C₃₋₆-cycloalkyl, OC₃-s-cycloalkyl, and OC₁₋₆-alkyl, wherein the alkyl groups may optionally be substituted with 1 to 3 halogen, and
C₃₋₆-cycloalkyl optionally substituted with 1 to 3 groups independently selected from the group consisting of halogen and C₁₋₆-alkyl optionally substituted with 1 to 3 halogen,
R2 is selected from the group consisting of H, C₁₋₆-alkyl such as —CH₃ and —CH₂CH₃, OCF₃, C₃₋₆-cycloalkyl, OC₁₋₆-alkyl, and OC₃₋₆-cycloalkyl,
R3 is selected from the group consisting of H, C₁₋₆-alkyl, C₃₋₆-cycloalkyl, and OC₃₋₆-cycloalkyl; wherein each of the C₁₋₆-alkyl, C₃₋₆-cycloalkyl, OC₃₋₆-cycloalkyl of the R³ group may be optionally substituted with one to three groups each independently selected from the group consisting of halogen, OH, OC₁₋₆alkyl, SC₁₋₆-alkyl, and N(C₁₋₆-alky)₂; and wherein one to three carbon atoms of the C₁₋₆-alkyl of the R3 group may optionally be replaced with one or two moieties selected from the group consisting of NH, N(C₁₋₆-alkyl), O, and S,
R4 and R5 are each independently selected from the group consisting of H and C₁₋₆-alkyl,
R3 and R4 can together with the atom to which they are attached join to form a 3 to 9-membered carbocyclyl ring which optionally may contain one to three heteroatoms selected from the group consisting of N, O, and S, or
R3 and R5 can together with the atoms to which they are attached join to form a 3 to 9-membered bicyclic ring which optionally may contain one to three heteroatoms selected from the group consisting of N, O, and S,
R6 is selected from the group consisting of H, C₁₋₆-alkyl, CN, CF₃, OCF₃, C₃₋₆-cycloalkyl, OC₁₋₆-alkyl, and OC₃₋₆-cycloalkyl,
R7 is selected from the group consisting of H and OC₁₋₆-alkyl,
or a pharmaceutically acceptable salt thereof together with one or more pharmaceutically acceptable carriers.

8. The method of claim 1, wherein the treatment is for a patient with bacterial or fungal severe sepsis or bacterial or fungal septic shock.

9. The method of claim 7, wherein the compound of formula (I), or a pharmaceutically acceptable salt thereof, is present in the pharmaceutical composition in the range from 0.1 to 90 wt.-% of the composition as a whole.

10. The method of claim 9, wherein the compound of formula (I), or a pharmaceutically acceptable salt thereof, is present in the pharmaceutical composition in the range from 0.1 to 50 wt.-% of the composition as a whole.
